(19) United States
(12) Patent Application Publication
(10) Pub. No.: US 2024/0189097 A1
(43) Pub. Date: Jun. 13, 2024

(54) DEVICES, ASSEMBLIES, AND METHODS FOR DELIVERING AGENTS

(71) Applicant: Boston Scientific Scimed, Inc., Maple Grove, MN (US)

(72) Inventors: Hannah REBAR, Brighton, MA (US); John B. GOLDEN, Norton, MA (US); Andrew PIC, Northboro, MA (US); Pauline Rosemary LIMBERG, Northborough, MA (US); Robert SEARS, Littleton, MA (US); Ryan EVERS, Billerica, MA (US); Alexandra HAUGEN, Westborough, MA (US); Adam Gregory McDERMOTT, Lincoln, MA (US)

(73) Assignee: Boston Scientific Scimed, Inc., Maple Grove, MN (US)

(21) Appl. No.: 18/530,637

(22) Filed: Dec. 6, 2023

Related U.S. Application Data
(60) Provisional application No. 63/386,444, filed on Dec. 7, 2022.

(51) Int. Cl. A61F 2/24

(52) U.S. Cl. CPC A61F 2/2403; A61F 2/2412; A61F 2/243; A61F 2/2436; A61F 2002/9505

(57) ABSTRACT

A valve assembly for a medical device that includes an enclosure to store an agent, a funnel coupled to the enclosure, and configured to receive the agent via an opening of the enclosure, and a valve fluidly coupled to the enclosure and the funnel. The valve is at least partially disposed between the enclosure and the funnel, and at least a portion of the agent is received on the valve. The valve is configured to move from a first position to a second position relative to the enclosure and the funnel to selectively release the agent from the enclosure into the funnel. In the first position, the valve is configured to close the opening and inhibit the agent from exiting the enclosure and entering the funnel. In the second position, the valve is configured to open the opening and release the agent from the enclosure for delivery into the funnel.

### CROSS-REFERENCE TO RELATED APPLICATIONS

This application claims the benefit of priority of U.S. Provisional Patent Application No. 63/386,444, filed on Dec. 7, 2022, the entirety of which is incorporated herein by reference

### TECHNICAL FIELD

Various aspects of this disclosure relate generally to devices and methods for delivering agents. More specifically, in embodiments, this disclosure relates to devices for delivery of powdered agents, such as hemostatic agents.

### BACKGROUND

In certain medical procedures, it may be necessary to minimize or stop bleeding internal to the body. For example, an endoscopic medical procedure may require hemostasis of bleeding tissue within the gastrointestinal tract, for example in the esophagus, stomach, or intestines. During an endoscopic procedure, a user inserts a sheath of an endoscope into a body lumen of a patient. The user utilizes a handle of the endoscope to control the endoscope during the procedure. Tools may be passed through a working channel of the endoscope via, for example, a port in the handle, to deliver treatment at the procedure site near a distal end of the endoscope. The procedure site is remote from the user.

To achieve hemostasis at the remote site, a hemostatic agent may be delivered by a delivery device inserted into the working channel of the endoscope. Agent delivery may be achieved, for example, through mechanical systems. Such systems, however, may require numerous steps or actuations to achieve delivery, may not achieve a desired rate of agent delivery or a desired dosage of agent, may result in the agent clogging portions of the delivery device, may result in inconsistent dosing of the agent, and/or may not result in the agent reaching the treatment site deep within the gastrointestinal tract. The current disclosure may solve one or more of these issues or other issues in the art.

### SUMMARY

Each of the aspects disclosed herein may include one or more of the features described in connection with any of the other disclosed aspects. Aspects of the disclosure relate to, among other things, systems, devices, and methods for delivering an agent to a target treatment site using a medical device including a valve assembly.

According to one example, a valve assembly may include an enclosure configured to store an agent, a funnel coupled to the enclosure and configured to receive the agent via an opening of the enclosure, and a valve fluidly coupled to the enclosure and the funnel. The valve may be at least partially disposed between the enclosure and the funnel, and at least a portion of the agent may be received on the valve, where the valve is configured to move from a first position to a second position relative to the enclosure and the funnel to selectively release the agent from the enclosure into the funnel. In the first position, the valve is configured to close the opening and inhibit the agent from exiting the enclosure and entering the funnel, and in the second position, the valve is configured to open the opening and release the agent from the enclosure for delivery into the funnel.

Any of the valve assemblies described herein may include any of the following features. The valve may include a body movably disposed within the opening, where, in the first position, at least a portion of the body is configured to extend into the opening, thereby fluidly decoupling the enclosure from the funnel, and in the second position, at least the portion of the body is configured to retract out of the opening, thereby fluidly coupling the enclosure with the funnel. The valve may include a hinge coupled to the body and a protrusion extending outwardly from the body, where the body is configured to pivot about the hinge when moving from the first position to the second position, and the protrusion is configured to extend into the enclosure when the body is in the first position and into the funnel when the body is in the second position. The valve may include a first body having one or more first ends and a second body having one or more second ends, where the second body is positioned opposite of the first body within the enclosure, where, in the first position, the first body is configured to engage the second body by interlocking the one or more first ends with the one or more second ends, and in the second position, the first body is configured to disengage the second body by separating the one or more first ends from the one or more second ends. The valve defines at least one wall of a plurality of walls of the enclosure, such that the at least one wall is configured to move relative to the remaining walls of the plurality of walls from the first position to the second position to form the opening. A channel may be positioned between the enclosure and the funnel, where the valve includes a rod that is movable relative to the channel, where, in the first position, the rod is configured to extend outwardly from the channel and into the opening of the enclosure, and in the second position, the rod is configured to retract into the channel and out of the opening. The valve may include a first expandable body positioned between the enclosure and the funnel, and a second expandable body positioned between the first expandable body and the funnel, where each of the first expandable body and the second expandable body include a center opening, where, in the first position, the first expandable body is at least partially unexpanded such that the center opening of the first expandable body is open to receive a first portion of the agent from the enclosure, and the second expandable body is expanded such that the center opening of the second expandable body is closed to inhibit the first portion of the agent received through the first expandable body from entering the funnel. The valve may include a first rotatable body and a second rotatable body. Each of the first rotatable body and the second rotatable body includes a plurality of projections, in the first position, a first projection of the first rotatable body and a first projection of the second rotatable body is configured to engage one another and form an interface for collecting a portion of the agent from the enclosure, and in the second position, the first projection of the first rotatable body and the first projection of second rotatable body are configured to disengage one another to open the interface between the first projection of the first rotatable body and the first projection of the second rotatable body for releasing the portion of the agent into the funnel. The valve may include a ring defining an opening and a plurality of leaves movably disposed within the opening. In the first position, each of the plurality of leaves may be extended radially inward from the ring and into the opening, such that each of the plurality of leaves is configured to engage one another and close the opening, and in the second position, each of the plurality of leaves is configured to retract radially outward from the opening such that each of the plurality of leaves is configured to disengage one another and open the opening. The valve may include a first flange, a second flange, and a flexible fabric coupled between the first flange and the second flange. The first flange and the second flange may collectively define a center opening and the flexible fabric is disposed within the center opening. In the first position, at least one of the first flange and the second flange is configured to move towards the other to transition the flexible fabric to a first configuration in which the flexible fabric closes the center opening, and in the second position, at least one of the first flange and the second flange is configured to move away from the other to transition the flexible fabric to a second configuration in which the flexible fabric opens the center opening. The valve may include a conduit defining a channel and a flexible sleeve disposed within the channel, where the conduit is configured to receive at least a portion of the agent from the enclosure. In the first position, the flexible sleeve is configured to move radially outward relative to the channel to a first configuration in which the flexible sleeve opens the conduit for receiving the agent from the enclosure; and in the second position, the flexible sleeve is configured to move radially inward relative to the channel to a second configuration in which the flexible sleeve closes the conduit and urges the agent out of the channel and towards the funnel. The valve may include a wire assembly coupled to the flexible sleeve, where the wire assembly is configured to move the flexible sleeve relative to the conduit between the first configuration and the second configuration. The valve may include a deformable body configured to extend into the opening of the enclosure, where, in the first position, the deformable body is configured to maintain a first shape that closes the opening of the enclosure, and in the second position, the deformable body is configured to transition to a second shape that opens the opening of the enclosure. The valve may include a deformable body coupled to the opening of the enclosure, where, in the first position, the deformable body is configured to cover the opening of the enclosure, and in the second position, the deformable body is configured to flex radially outward away from the opening of the enclosure, thereby allowing the agent to exit the enclosure and enter the funnel. The valve may include a seal including a plurality of slits, where, in the first position, each of the plurality of slits is configured to abut one another, thereby closing the opening of the enclosure, and in the second position, each of the plurality of slits is configured to extend outwardly away from one another such that the opening is formed through the seal for receiving the agent.

According to another example, a valve assembly for a medical device may include an enclosure defining a first chamber configured to store an agent and a second chamber configured to receive a pressurized fluid, and a valve disposed within the enclosure and positioned between the first chamber and the second chamber, where the valve is configured to fluidly couple the first chamber to the second chamber via an opening formed by the valve. The valve may be configured to move relative to the enclosure to a first position to close the opening between the first chamber and the second chamber, thereby inhibiting the agent from exiting the first chamber and entering the second chamber such that the pressurized fluid is not in fluid communication with the agent. The valve may also be configured to move relative to the enclosure to a second position to open the opening between the first chamber and the second chamber, thereby allowing the agent to exit the first chamber and enter the second chamber such that the pressurized fluid is in fluid communication with the agent.

Any of the valve assemblies described herein may include any of the following features. The valve may include a first expandable body positioned between the first chamber and the second chamber, and a second expandable body positioned between the first expandable body and the second chamber, where the opening is at least partially defined by each of the first expandable body and the second expandable body. In the first position, the first expandable body is at least partially compressed and the second expandable body is expanded, such that a first portion of the agent from the first chamber is received through the first expandable body and not through the second expandable body. In the second position, the first expandable body is expanded and the second expandable body is at least partially compressed, such that the first portion of the agent received through the first expandable body is received through the second expandable body and released into the second chamber via the opening. In the first position, the second expandable body is configured to inhibit the first portion of the agent received through the first expandable body from entering the second chamber via the opening. In the second position, the first expandable body is configured to inhibit a second portion of the agent from the first chamber from entering through the second expandable body via the opening. The valve includes an iris valve formed of a flexible fabric that is configured to move laterally relative to the enclosure to form the opening between the first chamber and the second chamber.

According to another example, a method for delivering an agent from a medical device, the medical device including an enclosure having a first chamber storing the agent and a second chamber that is in fluid communication with a pressurized fluid, may include actuating the medical device to deliver the pressurized fluid to the second chamber of the enclosure, moving a valve of the medical device from a first position to a second position in response to the pressurized fluid being delivered to the second chamber, where, in the first position, the valve is configured to seal the first chamber from the second chamber such that the agent is inhibited from exiting the first chamber, and in the second position, the valve is configured to unseal the first chamber from the second chamber such that the agent is permitted to exit the first chamber and enter the second chamber, agitating the agent with the pressurized fluid within the second chamber, thereby forming a mixture of the agent and the pressurized fluid, and guiding the mixture of the agent and the pressurized fluid through an outlet of the medical device.

It may be understood that both the foregoing general description and the following detailed description are exemplary and explanatory only and are not restrictive of the invention, as claimed. As used herein, the terms “comprises,” “comprising,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “diameter” may refer to a width where an element is not circular. The term “top” refers to a direction or side of a device relative to its orientation during use, and the term “bottom” refers to a direction or side of a device relative to its orientation during use that is opposite of the “top.” The term “exemplary” is used in the sense of “example,” rather than “ideal.” The term “approximately,” or like terms (e.g., “substantially”), includes values+/−10% of a stated value.

### BRIEF DESCRIPTION OF THE DRAWINGS

The accompanying drawings, which are incorporated in and constitute a part of this specification, illustrate aspects of this disclosure and together with the description, serve to explain the principles of the disclosure.

FIG. 1 shows an exemplary delivery device according to some embodiments.

FIG. 2A shows a first position of an exemplary valve assembly of the delivery device of FIG. 1 according to some embodiments.

FIG. 2B shows a second position of the valve assembly of FIG. 2A according to some embodiments.

FIG. 3A shows a first position of another exemplary valve assembly of the delivery device of FIG. 1 according to some embodiments.

FIG. 3B shows a second position of the valve assembly of FIG. 3A according to some embodiments.

FIG. 4 shows an exploded view of another exemplary valve assembly of the delivery device of FIG. 1 according to some embodiments.

FIG. 5A shows a first position of the valve assembly of FIG. 4 according to some embodiments.

FIG. 5B shows a second position of the valve assembly of FIG. 4 according to some embodiments.

FIG. 6A shows a first position of another exemplary valve assembly of the delivery device of FIG. 1 according to some embodiments.

FIG. 6B shows a second position of the valve assembly of FIG. 6A according to some embodiments.

FIG. 7A shows a first position of another exemplary valve assembly of the delivery device of FIG. 1 according to some embodiments.

FIG. 7B shows a second position of the valve assembly of FIG. 7A according to some embodiments.

FIG. 8 shows an exploded view of another exemplary valve assembly of the delivery device of FIG. 1 according to some embodiments.

FIG. 9A shows a first position of the valve assembly of FIG. 8 according to some embodiments.

FIG. 9B shows a second position of the valve assembly of FIG. 8 according to some embodiments.

FIG. 10A shows a first position of another exemplary valve assembly of the delivery device of FIG. 1 according to some embodiments.

FIG. 10B shows a second position of the valve assembly of FIG. 10A according to some embodiments.

FIG. 11A shows a first position of another exemplary valve assembly of the delivery device of FIG. 1 according to some embodiments.

FIG. 11B shows a second position of the valve assembly of FIG. 11A according to some embodiments.

FIG. 12A shows a first position of another exemplary valve assembly of the delivery device of FIG. 1 according to some embodiments.

FIG. 12B shows a second position of the valve assembly of FIG. 12A according to some embodiments.

FIG. 13A shows a first position of another exemplary valve assembly of the delivery device of FIG. 1 according to some embodiments.

FIG. 13B shows a second position of the valve assembly of FIG. 13A according to some embodiments.

FIG. 14A shows a first position of another exemplary valve assembly of the delivery device of FIG. 1 according to some embodiments.

FIG. 14B shows a second position of the valve assembly of FIG. 14A according to some embodiments.

FIG. 15A shows a first position of another exemplary valve assembly of the delivery device of FIG. 1 according to some embodiments.

FIG. 15B shows a second position of the valve assembly of FIG. 14A according to some embodiments.

FIG. 16 shows an exploded view of another exemplary valve assembly of the delivery device of FIG. 1 according to some embodiments.

FIG. 17A shows a first position of the valve assembly of FIG. 16 according to some embodiments.

FIG. 17B shows a second position of the valve assembly of FIG. 16 according to some embodiments.

### DETAILED DESCRIPTION

Embodiments of this disclosure relate to dispensing devices having valve assemblies for selectively releasing an agent (e.g., a powdered agent) to a site of a medical procedure. The valve assembly may include at least one movable body, which may be moved from a first position to a second position to form an opening in an enclosure of the dispensing device that stores the agent, in order to selectively release the agent from the enclosure. The enclosure may store the agent, and may not be in fluid communication with a pressurized medium source (e.g., a gas canister). Once released from the enclosure, the agent may be moved to an area of the dispensing device in which the agent encounters the pressurized fluid (e.g., a gas). The agent may be in fluid communication with the pressurized fluid through an outlet and/or opening formed by the valve assembly. Accordingly, when the agent is selectively moved into fluid communication with the pressurized fluid source, when the valve assembly forms the opening in the enclosure, the agent may be agitated by the fluid prior to delivery to a target site of the medical procedure. Aspects of the dispensing device and valve assembly, such as the at least one movable body, may facilitate a controlled fluidization of the agent with the flow of pressurized fluid prior to the agent being delivered, which may assist in selectively controlling the flow of agent out of the dispensing device to help to prevent or minimize clogging during delivery.

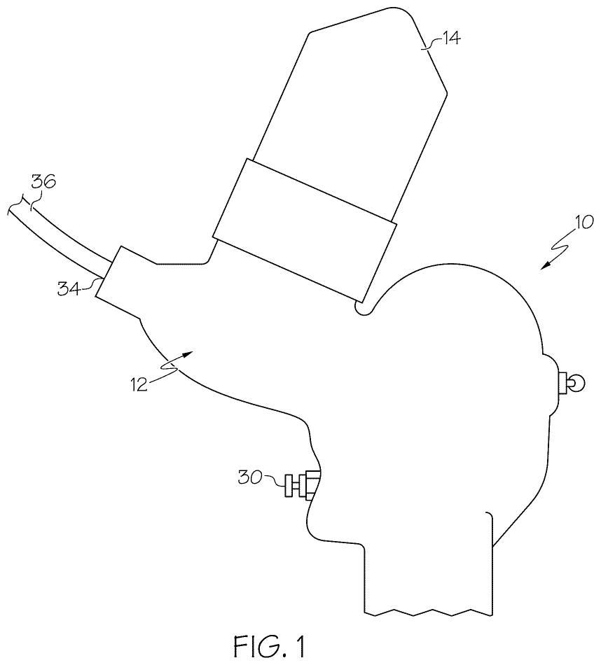

FIG. 1 shows a delivery system 10, which may be a powder delivery system. Delivery system 10 may include a handle body 12. Handle body 12 may include, or may be configured to receive, an enclosure 14 (or other source or container) storing a material (e.g., a powdered agent). Enclosure 14 may be coupled to handle body 12 for providing the agent to handle body 12, or a lid/enclosure of the agent may be screwed onto, or otherwise coupled to, enclosure 14 for supplying the agent to enclosure 14. The agent may be, for example, a powdered agent, such as a hemostatic agent. The agent may alternatively be another type of agent or material, or form of agent (e.g., a liquid or gel agent), and may have any desired function. Enclosure 14 may be removably attached to other components of delivery system 10, including components of handle body 12.

Handle body 12 may have a variety of features, to be discussed in further detail herein. U.S. patent application Ser. No. 16/589,633, filed Oct. 1, 2019, published as U.S. Patent Application Publication No. 2020/0100986 A1 on Apr. 2, 2022, the disclosure of which is hereby incorporated by reference in its entirety, discloses features of exemplary delivery devices and systems. The features of this disclosure may be combined with any of the features described in the above-referenced application. The features described herein may be used alone or in combination and are not mutually exclusive. Like reference numbers and/or terminology are used to denote similar structures, when possible.

Still referring to FIG. 1, delivery system 10 may include an actuation mechanism 30 used to activate flow of a pressurized fluid (e.g., gas) from a pressurized medium source in fluid communication with delivery system 10. Actuation mechanism 30 may be selectively actuated (e.g., manually depressible) or otherwise moved or actuated to control delivery of a material (e.g., a powdered agent) and pressurized fluid. The pressurized fluid alone, or a combination of a powdered agent and fluid, may be delivered from an outlet 34 of handle body 12. Outlet 34 may be in fluid communication with a delivery conduit, for example a catheter 36 or another component for delivering the combination of agent and fluid to a desired location within a body lumen of a patient. Delivery system 10 may include a valve assembly for selectively controlling the release of the powdered agent and/or the pressurized fluid within enclosure 14.

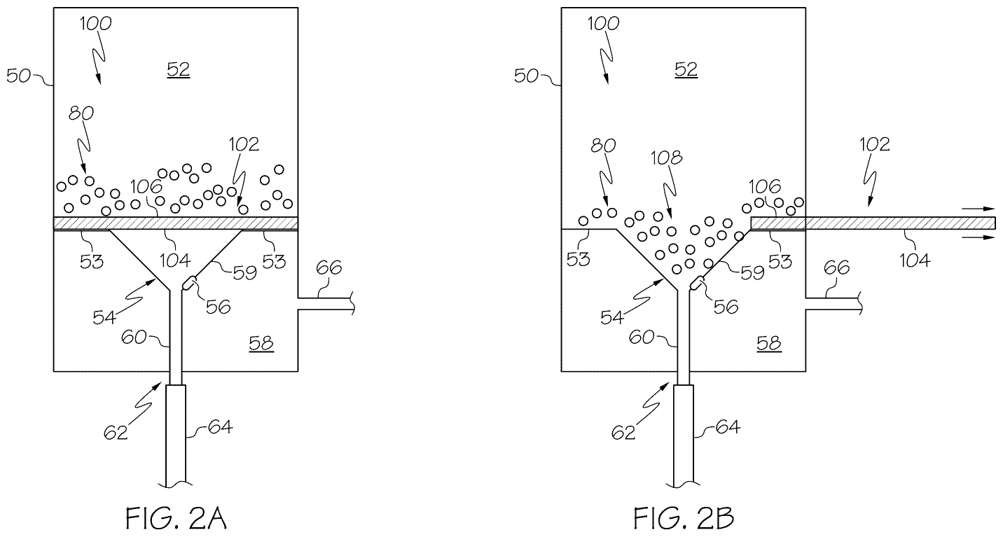

FIGS. 2A and 2B show aspects of an exemplary valve assembly 100, according to some embodiments. Valve assembly 100 may be housed within handle body 12 of delivery system 10, and selectively actuated by actuation mechanism 30. In some embodiments, actuation mechanism 30 may be coupled to valve assembly 100 by a mechanical connection. For example, the mechanical connection may include a cable, a wire, a rod, or various other suitable mechanisms for connecting one or more internal components of valve assembly 100 to actuation mechanism 30. However, in some embodiments, actuation mechanism 30 may be coupled to valve assembly 100 by a pneumatic connection. Although not shown, actuation mechanism 30 may include one or more other actuation elements, such as, for example, a trigger, a button, a slider, a lever, a knob, a dial, and various other suitable actuators. As described herein, actuation of actuation mechanism 30 may provide for a corresponding movement of valve assembly 100, thereby controlling a delivery of the agent through valve assembly 100.

Valve assembly 100 may include a container 50 (e.g., an enclosure), which may be configured to store an agent 80 (e.g. a powder), for example a hemostatic agent. It should be appreciated that the depiction of agent 80 throughout the figures is merely illustrative such that a size, a shape, a configuration, a position, an arrangement, a quantity (volume), and/or various other characteristics of agent 80 is exemplary and may vary from that shown and described herein without departing from a scope of this disclosure. As shown in FIGS. 2A and 2B, for example, container 50 may include a first cavity or chamber 52 and a second cavity or chamber 58 separated by a funnel 54 disposed therebetween. First chamber 52 may be positioned above second chamber 58, and may be configured to store agent 80. In some embodiments, funnel 54 may be disposed within second chamber 58 beneath first chamber 52, and funnel 54 may include an upper rim or wall 53. As described herein, funnel 54 may be in fluid communication with first chamber 52 in response to valve assembly 100 moving from a first position (FIG. 2A) to a second position (FIG. 2B).

Funnel 54 may include a sidewall 59 extending downward from upper wall 53, and at least a portion 56 of sidewall 59 may be sintered and/or porous (hereinafter referred to as porous portion 56). For example, porous portion 56 of sidewall 59 may include a plurality of pores and/or passages formed between an outer surface of sidewall 59 and an inner surface of sidewall 59. In some embodiments, the pores of porous portion 56 may have sizes ranging from between approximately 40 microns and 150 microns (e.g., 100 microns). By way of illustrative example, particle sizes of agent 80 may range from approximately 200 microns to 600 microns (e.g., 320 microns to 400 microns). As described below, porous portion 56 may be configured to allow a pressurized fluid received within second chamber 58 to pass through funnel 54 for agitating agent 80 received therein. Funnel 54 may include a channel 60 extending downwardly from sidewall 59, and channel 60 may be configured to guide a mixture of the pressurized fluid and agent 80 out of valve assembly 100 via an outlet 62 of container 50. In some embodiments, a fluid inlet 66 may be coupled to second chamber 58. Fluid inlet 66 may be positioned adjacent to funnel 54, and particularly facing porous portion 56. Valve assembly 100 may include a tubing 64 coupled to channel 60, and tubing 64 may be configured to receive a mixture of pressurized fluid and agent 80 for delivering out of container 50 at outlet 62.

In some embodiments, funnel 54 may be substantially conical or funnel-shaped to reduce packing and/or clogging of agent 80 received therein. For example, a proximal (upper) end funnel 54 may have a greater diameter relative to a distal (lower) end of funnel 54. Sidewall 59 may be angled and/or tapered such that funnel 54 may define a smaller cavity in a distal (lower) direction adjacent to channel 60.

Valve assembly 100 may include a movable body 102 (e.g. a door, a wall, an impediment, etc.) disposed within container 50 between first chamber 52 and second chamber 58. For example, as shown in FIG. 2A, movable body 102 may be movably coupled to an interior surface and/or wall of container 50. In some examples, movable body 102 may be movably coupled to upper wall 53. Movable body 102 may be configured to close an opening 108 (see FIG. 2B) between first chamber 52 and second chamber 58 when movable body 102 is in a first position. In other words, movable body 102 may extend into container 50, along and/or adjacent to upper wall 53, thereby closing opening 108 such that an upper surface 106 of movable body 102 may be positioned adjacent to first chamber 52, and a lower surface 104 of movable body 102 may be positioned adjacent to funnel 54 and/or second chamber 58. Accordingly, first chamber 52 may be fluidly decoupled from funnel 54 by movable body 102 such that agent 80 stored within first chamber 52 is inhibited from flowing out of first chamber 52 and into funnel 54 and/or second chamber 58 by movable body 102.

As shown in FIG. 2B, for example, movable body 102 may be moved to a second position relative to container 50, such as in response to actuation of actuation mechanism 30. When in the second position, at least a portion of movable body 102 may be translated and/or retracted away from a location of opening 108 (e.g. a central region of container 50) such that at least a portion of opening 108 is opened, thereby fluidly coupling first chamber 52 with funnel 54 and/or second chamber 58. In this instance, agent 80 stored within first chamber 52 may be released into funnel 54 via opening 108. Movable body 102 may have any size and/or shape suitable to close opening 108. For example, movable body 102 may have a circular, a rectangular, a squared, a convex, a concave, a conical, a flat (planar), a claw-shaped, a stopper-shaped, and/or various other suitable shapes and/or configurations without departing from a scope of this disclosure. In some embodiments, movable body 102 may have the same size and/or shape as opening 108.

In some embodiments, a pressurized fluid may be released into second chamber 58 of container 50, beneath movable body 102, via fluid inlet 66. Porous portion 56 may be configured to permit the fluid received from fluid inlet 66 to flow through sidewall 59 to agitate agent 80 received in funnel 54. Accordingly, the pressurized fluid may flow into second chamber 58 and through porous portion 56 to mix with agent 80 within funnel 54. Upon agitation of agent 80, funnel 54 may be configured to guide a mixture of the agitated agent 80 and the pressurized fluid into channel 60 and towards outlet 62.

The pores of porous portion 56 may cause the pressurized fluid flowing through second chamber 58 to enter funnel 54 at a wide variety of vectors, including angles, velocities, and/or pressures at the same time. The fluid exiting funnel 54 may have a turbulent flow pattern (e.g., a radial pattern). The varying vectors with which fluid enters funnel 54 may cause agent 80 entering funnel 54 from first chamber 52 to become fluidized. The turbulent flow of fluid (which may result in fluidization, such as a liquid sand effect, of agent 80) may aid in guiding an agitated agent 80 through outlet 62, and may prevent or minimize clogging of agent 80 within first chamber 52. Fluidization of agent 80 may also assist in breaking up agglomerates of agent 80 prior to delivery through outlet 62.

Still referring to FIG. 2B, movable body 102 may be maintained at the second position as long as actuation mechanism 30 is actuated, such that agent 80 stored in first chamber 52 may be continuously released from first chamber 52 and into funnel 54 via opening 108. Alternatively, in other embodiments, movable body 102 may be configured to transition from the first position to the second position in predetermined intervals for as long as actuation mechanism 30 is actuated. The predetermined intervals may have a duration between about 1 second to about 5 seconds. It should be appreciated that such exemplary intervals are merely provided for illustrative purposes, such that the predetermined intervals may be longer than 5 seconds and/or shorter than 1 second without departing from a scope of this disclosure. In some embodiments, the predetermined interval may be at least partially determined based on a medical procedure. In other embodiments, movable body 102 may be configured to automatically return to first position after a predetermined duration. For example, once actuation mechanism 30 is actuated, movable body 102 may be configured to move to the second position for a predetermined amount of time before returning to the first position, thereby allowing only a predetermined volume of agent 80 to be released from first chamber 52 into funnel 54. Accordingly, as only a predetermined amount of agent 80 is delivered per each actuation of delivery system 10, valve assembly 100 may allow for greater control over the amount of agent 80 delivered to a treatment site, which may improve a treatment procedure of a patient.

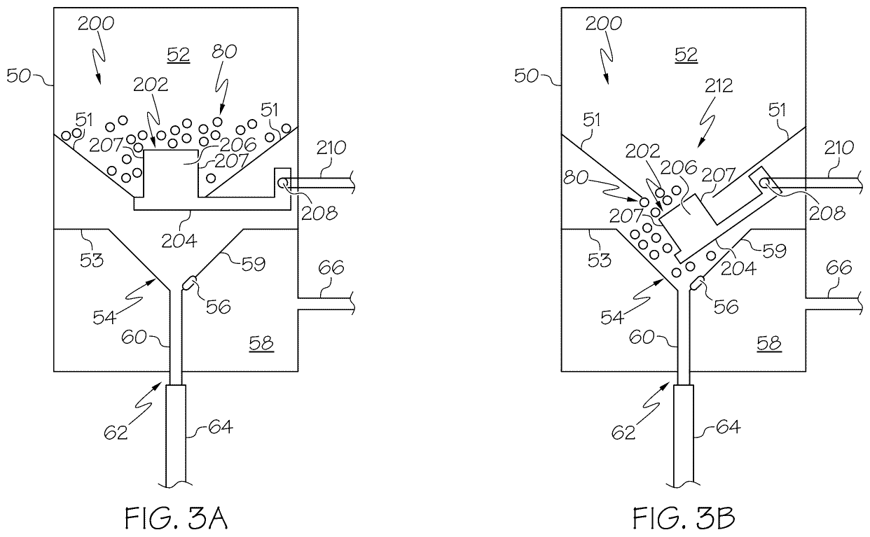

As described herein, delivery system 10 may include several different configurations of a valve assembly that is positioned within and/or coupled to container 50 and in fluid communication with one or more of first chamber 52 and second chamber 58. For example, as shown in FIGS. 3A and 3B, another exemplary valve assembly 200 is depicted. Valve assembly 200 may be configured and operable like valve assembly 100 except for the differences explicitly described herein. Accordingly, like reference numerals are used to identify like components. Valve assembly 200 may include a movable body 202 (e.g., a door, a wall, a stopper, a plunger, etc.), which may be configured to act as a stopper between first chamber 52 and second chamber 58. In some embodiments, movable body 202 may include a lower wall or surface 204 and a protrusion 206 extending outwardly from lower surface 204. Protrusion 206 may be configured to fit within an opening 212 (see FIG. 3B) of container 50. Opening 212 may be disposed between a pair of inner sidewalls 51 of container 50 in first chamber 52, such that agent 80 stored within first chamber 52 may be at least partially disposed along inner sidewalls 51. In the example, inner sidewalls 51 may be angled relative to one another to facilitate movement of agent 80 towards opening 212.

Still referring to FIG. 3A, protrusion 206 may have any size and/or shape suitable to close opening 212. For example, a size and/or a shape of protrusion 206 may be adapted to a size (e.g. a diameter) and/or shape (e.g. circular) of opening 212, such that protrusion 206 may be configured to form a fluid tight seal with the pair of inner sidewalls 51 when received within opening 212. Additionally, the size and/or shape of protrusion 206 may inhibit bridging of agent 80 within container 50 by extending into first chamber 52 when valve assembly 200 is in the first position. For example, in some embodiments, movable body 202 may include one or more walls 207 defining protrusion 206, which may be angled to allow for agent 80 to more easily slide down the sides and/or exterior of protrusion 206, and may be sized and/or shaped to occupy a volume of first chamber 52 when valve assembly 200 is in the first position. In this instance, movable body 202 may be configured to inhibit first chamber 52 from being over-filled with agent 80.

Valve assembly 200 may include an actuating member 210 coupled to movable body 202 for selectively controlling movement of movable body 202, and particularly protrusion 206, when transitioning between the first position and the second position. For example, actuating member 210 may include a lever, a rod, a cable, a wire, and/or various other suitable mechanisms for moving movable body 202 relative to container 50. In the example, actuating member 210 may be movably coupled to a first end or portion of movable body 202 at a pivot joint 208 (e.g. a hinge), such that movement of actuating member 210 in a first direction (e.g., lateral) may provide a corresponding movement of movable body 202 in a second direction (e.g. vertical) that is different than the first direction.

Still referring to FIG. 3A, with movable body 202 in the first position, protrusion 206 may extend into first chamber 52 such that protrusion 206 may inhibit fluid communication between first chamber 52 and second chamber 58. In this instance, lower surface 204 of movable body 202 may be positioned adjacent to funnel 54, and at least a portion of movable body 202 (e.g. protrusion 206) may be in contact with the pair of inner sidewalls 51, thereby fluidly sealing first chamber 52 from second chamber 58.

As shown in FIG. 3B, movable body 202 may be configured to move to a second position by pivoting about pivot joint 208 in response to actuation (e.g., translation) of actuating member 210, which may be coupled to actuation mechanism 30. For example, upon moving actuating member 210 relative to container 50, movable body 202 (and particularly protrusion 206) may be configured to move (e.g. pivot) away from first chamber 52 such that protrusion 206 may disengage the pair of inner sidewalls 51. In this instance, movable body 202 (and particularly protrusion 206) may be removed from opening 212, thereby fluidly coupling first chamber 52 with second chamber 58 and funnel 54 disposed therein. In this instance, agent 80 stored within first chamber 52 may be released into funnel 54 via opening 212 and agitated with the pressurized fluid received in second chamber 58 (and particularly funnel 54) via fluid inlet 66 as described in detail above.

In other embodiments, movable body 202 may be configured to move in various other directions than those shown and described herein without departing from a scope of this disclosure. For example, movable body 202 may be configured to move laterally or vertically relative to container 50, and/or swing about pivot joint 208 in one or more directions. In further embodiments, movable body 202 may be oriented in various other arrangements, such as with protrusion 206 disposed within second chamber 58 and/or adjacent to funnel 54 when valve assembly 200 is in the first position.

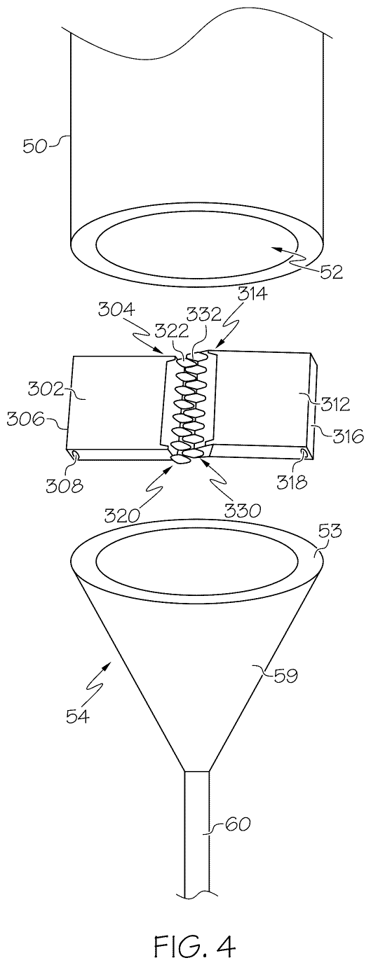

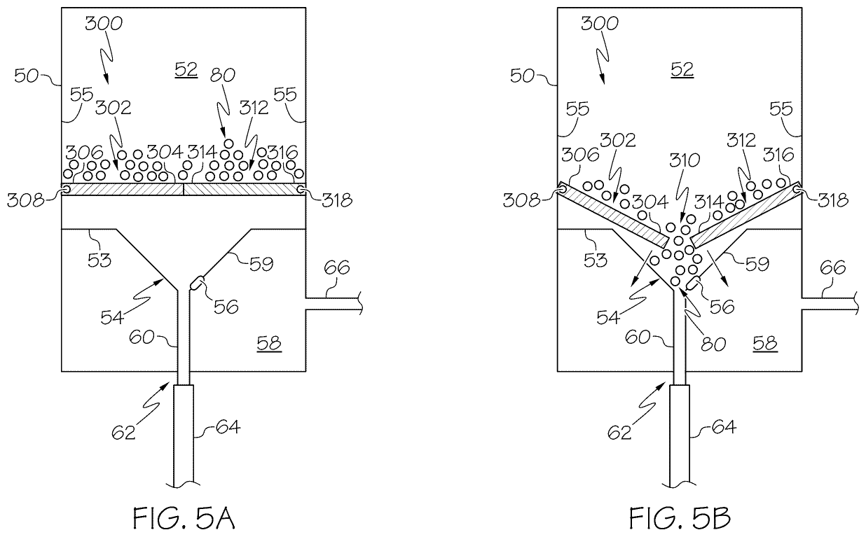

FIGS. 4-5B show another exemplary valve assembly 300 that may be substantially similar to valve assembly 100 except for the differences explicitly noted herein. Accordingly, like reference numerals may be used to identify like components. Valve assembly 300 may include a first movable body 302 and a second movable body 312. Movable bodies 302, 312 may include, for example, a pair of doors, a pair of walls, a pair of jaws or claws, a pair of leaves, and/or various other suitable mechanisms.

As shown in FIG. 4, in some embodiments, first movable body 302 and second movable body 312 may each include an interlocking mechanism 320, 330 at a first end 304 of first movable body 302 and at a first end 314 of second movable body 312, respectively. First interlocking mechanism 320 may be configured to interlock with second interlocking mechanism 330 when first movable body 302 engages second movable body 312. In some embodiments, each of first and second interlocking mechanisms 320, 330 may include a plurality of teeth 322, 332 or other component (e.g., fingers, leaves, claws, arms, etc.) configured to interlace and/or interlock with the corresponding interlocking mechanism 320, 330. It should be appreciated that a size, a shape, a quantity, and/or a configuration of teeth 322, 332 may vary from that shown and described herein without departing from a scope of this disclosure.

In response to first movable body 302 engaging second movable body 312 (or vice versa), interlocking mechanisms 320, 330 may be configured to close an opening between first movable body 302 and second movable body 312 such that the agent stored within first chamber 52 is inhibited from being released into funnel 54 and/or second chamber 58. Stated differently, the plurality of teeth 322 may interface with the plurality of teeth 332 when first end 304 is positioned adjacent to first end 314, thereby interlocking teeth 322 with teeth 332. In this instance, first movable body 302 and second movable body 312 may be configured to form a fluid tight seal between first chamber 52 and second chamber 58 and/or funnel 54.

First movable body 302 may include a second end 306 that is opposite of first end 304, and second movable body 312 may include a second end 316 that is opposite of first end 314. Second end 306 may include a pivot joint 308 such that first movable body 302 may be configured to move (e.g. pivot) about pivot joint 308, such as when valve assembly 300 transitions from the first position to the second position. Second end 316 may include a pivot joint 318 such that second movable body 312 may be configured to move (e.g. pivot) about pivot joint 318, such as when valve assembly 300 transitions from the first position to the second position. Each of second end 306 and second end 316 may be coupled to an interior surface and/or wall of container 50, such that pivot joints 308, 318 may be positioned adjacent to the interior surface and/or wall of container 50.

Alternatively, in other embodiments as shown in FIGS. 5A and 5B, first movable body 302 and second movable body 312 may be formed without interlocking mechanisms. Instead, the interface formed between first end 304 of first movable body 302 and first end 314 of second movable body 312, as shown in FIG. 5A, may be configured close first chamber 52 from second chamber 58, thereby fluidly decoupling first chamber 52 and funnel 54. Accordingly, agent 80 stored within first chamber 52 may be inhibited from being released into funnel 54 when valve assembly 300 is in the first position. In the example, second end 306 may be coupled to an inner wall 55 of container 50 at pivot joint 308, and second end 316 may be coupled to an opposing inner wall 55 of container 50 at pivot joint 318.

As shown in FIG. 5B, in some embodiments, when valve assembly 300 is moved to the second position, first movable body 302 and second movable body 312 may be moved (pivoted) away from each other, such as either in an upward or a downward direction, via pivot joints 308, 318 disposed at the respective second ends 306, 312. Pivoting first movable body 302 and second movable body 312 away from one another may form an opening 310 between first end 304 of first movable body 302 and first end 314 of second movable body 312, thereby fluidly coupling first chamber 52 with second chamber 58 and/or funnel 54. Accordingly, agent 80 stored within first chamber 52 may be released into funnel 54 via opening 310 and agitated with the pressurized fluid received within second chamber 58 (and particularly funnel 54) via fluid inlet 66 as described in detail above.

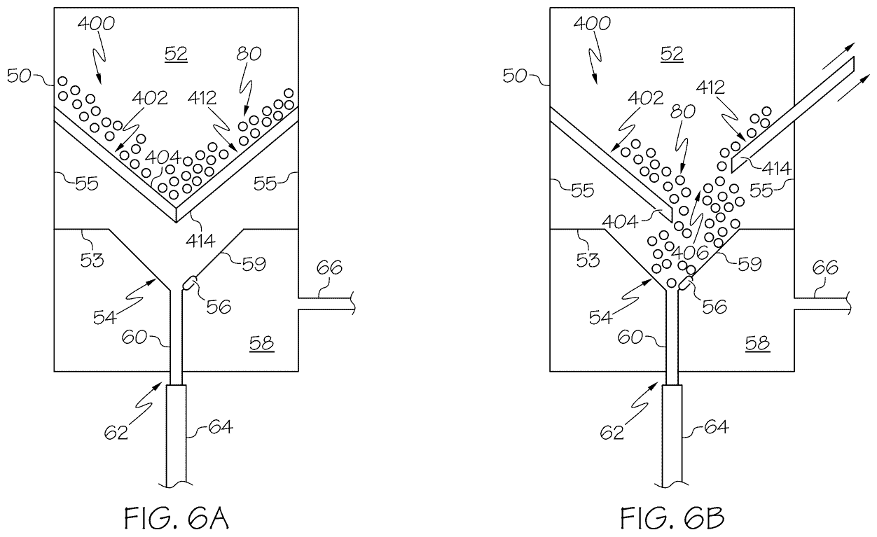

FIGS. 6A and 6B show aspects of another exemplary valve assembly 400 that is substantially similar to valve assembly 100 shown and described above except for the differences explicitly noted herein. Accordingly, like reference numerals are used to identify like components. For example, as shown in FIG. 6A, valve assembly 400 may include a first body 402 that is fixedly coupled to an inner wall 55 of container 50, and a second body 412 that is movably coupled to an opposing inner wall 55 of container 50. In the example, each of first body 402 and second body 412 may be positioned at an angle with respect to inner walls 55 of container 50. In other examples, bodies 402, 412 may be aligned parallel, perpendicular, and/or transverse to inner walls 55. First body 402 and second body 412 may collectively define interior sidewalls of first chamber 52 such that first body 402 and second body 412 may collect at least a portion of agent 80 stored within first chamber 52 thereon.

It should be appreciated that first body 402 and second body 412 may have various suitable sizes, shapes, configurations, and/or arrangements relative to one another. For example, first body 402 and second body 412 may include sidewalls within first chamber 52 defining a common junction point. In some embodiments, first body 402 and second body 412 may define a two-surface and/or three-surface junction, such as an edge of a triangular prism, a point of a conical surface, a corner of a squared, rectangular, or cubed interface, and/or a flat or curved surface of a cylindrical chamber.

Referring specifically to FIG. 6A, first body 402 may include a first end 404 and second body 412 may include a first end 414. When valve assembly 400 is in a first position, the interface formed between first end 404 of first body 402 and first end 414 of second body 412 may be configured to form a barrier between first chamber 52 and second chamber 58, thereby fluidly decoupling first chamber 52 from funnel 54. Accordingly, agent 80 stored within first chamber 52 may be inhibited from being released into funnel 54 when valve assembly 400 is in the first position.

When valve assembly 400 is moved to the second position, as shown in FIG. 6B, second body 412 may be moved relative to container 50 and away from first body 402, thereby disengaging first end 414 from first end 404. In some embodiments, second body 412 may be moved within and/or outside of container 50, such as in response to actuation of actuation mechanism 30, thereby forming an opening 406 between first end 404 of first body 402 and first end 414 of second body 412. In some embodiments, actuation of actuation mechanism 30 may cause second body 412 to be pulled away from first body 402 while first body 402 remains stationary relative to container 50. Once opening 406 is formed, first chamber 52 may be fluidly coupled with second chamber 58 and funnel 54 disposed therein, such that agent 80 stored within first chamber 52 may be released into funnel 54 via opening 406 and agitated with the pressurized fluid received within second chamber 58 (and particularly funnel 54) via fluid inlet 66 as described in detail above.

In some embodiments, second body 412 may be biased to the first position such that second body 412 may be maintained in the first position absent an opposing force applied to second body 412, such as in response to an actuation of actuation mechanism 30. For example, valve assembly 400 may include a biasing mechanism (not shown) configured to bias second body 412 to the first position in order to close opening 406. Once actuation mechanism 30 is disengaged, the biasing mechanism (e.g., a spring) may be configured to return second body 412 to the first position, thereby closing opening 406.

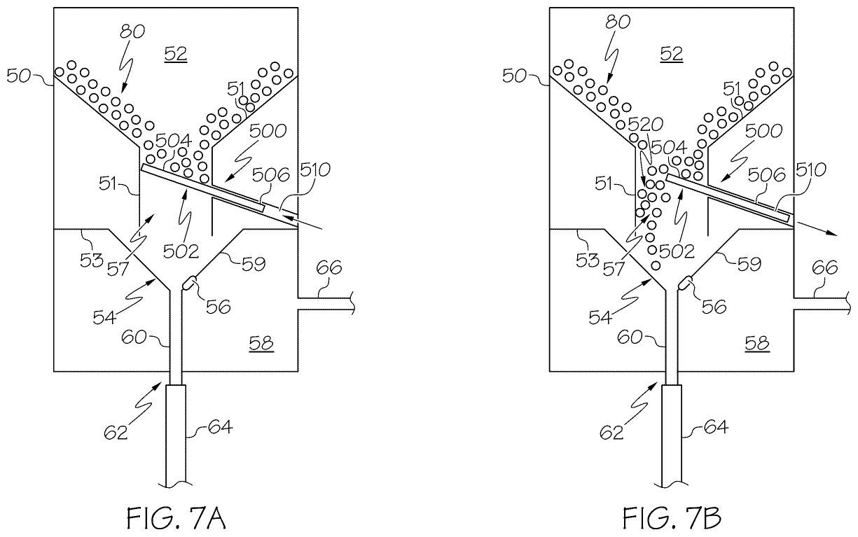

Referring now to FIGS. 7A and 7B, another exemplary valve assembly 500 is depicted. Valve assembly 500 may be substantially similar to valve assembly 100 except for the differences explicitly noted herein. Accordingly, like reference numerals are used to identify like components. For example, valve assembly 500 may include a movable body 502 (e.g., a rod, a door, a wall, a wire, a plunger, a stopper, etc.) having a longitudinal length defined between a first end 504 and a second end 506 that is opposite of first end 504. Container 50 may include a channel 57 defined between the pair of inner sidewalls 51, and at least one recess 510 formed within at least one of the inner sidewalls 51. Movable body 502 may be at least partially disposed within recess 510, and particularly second end 506 of movable body 502 may be received therein. Movable body 502 may be configured to selectively extend outward from recess 510 and across channel 57, such that movable body 502 may positioned between first chamber 52 and second chamber 58 (and/or funnel 54) when disposed within channel 57. In this instance, movable body 502 may be configured to form a fluid tight seal between first chamber 52 and second chamber 58.

In some embodiments, movable body 502 may extend laterally between inner sidewalls 51 and across channel 57 relative to upper wall 53. In other embodiments, movable body 502 may extend between inner sidewalls 51 and across channel 57 at an angle relative to upper wall 53. It should be appreciated that movable body 502 may have various suitable sizes, shapes, and/or configurations than those shown and described herein without departing from a scope of this disclosure.

Referring specifically to FIG. 7A, when valve assembly 500 is in the first position, movable body 502 may extend across an entire width of channel 57 such that first end 504 may contact the inner sidewall 51 opposing recess 510. Accordingly, first chamber 52 may be fluidly decoupled from second chamber 58 and/or funnel 54 when movable body 502 is disposed within channel 57 and in the first position.

Referring now to FIG. 7B, when valve assembly 500 is moved to the second position, such as in response to an actuation of actuation mechanism 30, movable body 502 may be at least partially retracted out of channel 57 and into recess 510. For example, second end 506 of movable body 502 may be moved further into recess 510, thereby pulling first end 504 out of channel 57. When movable body 502 is retracted from channel 57, first end 504 may disengage the inner sidewall 51 opposing recess 510, such that an opening 520 may be formed between first end 504 of movable body 502 and the opposing inner sidewall 51. In this instance, movable body 502 may be configured to fluidly couple first chamber 52 and second chamber 58 (and/or funnel 54) such that agent 80 stored within chamber 52 may be released into funnel 54 via opening 520 and agitated with the pressurized fluid received within second chamber 58 (and particularly funnel 54) via fluid inlet 66. In some embodiments, movable body 502 may remain positioned at least partially within channel 57 and opening 520 when valve assembly 500 is in the second position. In this instance, movable body 502 may be configured to break up blockages of agent 80 in first chamber 52 as agent 80 moves through opening 520 and encounters first end 504 within channel 57.

In some embodiments, movable body 502 may be biased to the first position such that movable body 502 may be maintained in the first position absent an opposing force applied to movable body 502, such as in response to an actuation of actuation mechanism 30. For example, valve assembly 500 may include a biasing mechanism (not shown) configured to bias movable body 502 to the first position in order to close opening 520. Actuation of actuation mechanism 30 may counteract a biasing force exerted by the biasing mechanism (e.g., a spring) to move movable body 502 to the second position. Once actuation mechanism 30 is disengaged, the biasing mechanism may be configured to return movable body 502 to the first position, thereby closing opening 520.

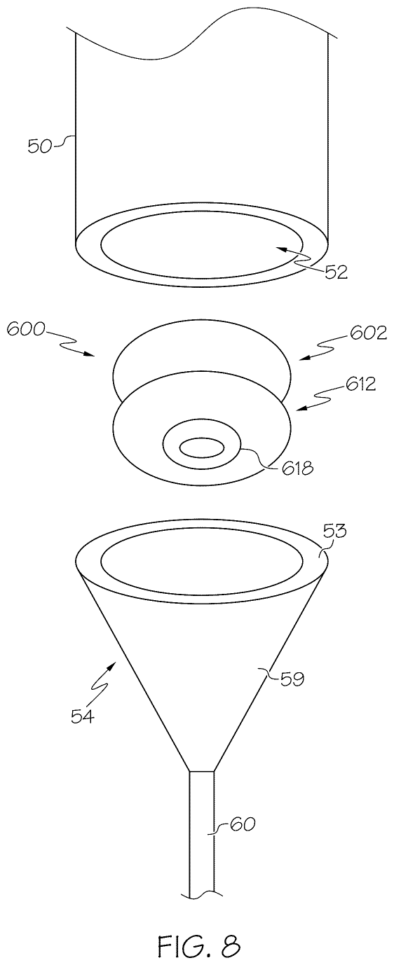

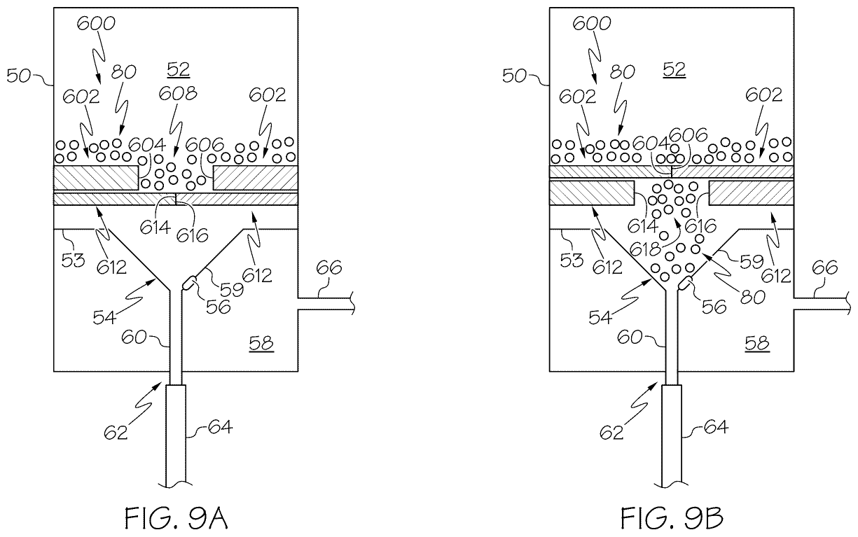

FIGS. 8-9B show aspects of another exemplary valve assembly 600 that is substantially similar to valve assembly 100 except for the differences explicitly described herein. Accordingly, like reference numerals are used to identify like components. Valve assembly 600 may include a first expandable body 602 and a second expandable body 612, each of which may be expanded independently relative to one another. In some embodiments, first expandable body 602 and second expandable body 612 may include balloons configured to be selectively inflated and/or deflated independent of one another. Expandable bodies 602, 612 may be selectively inflated and/or deflated via a pneumatic, a hydraulic, and/or a mechanical mechanism. For example, expandable bodies 602, 612 may be inflated and/or deflated with a pressurized medium upon actuation of an actuator, such as, for example, actuation mechanism 30.

In some embodiments, expandable bodies 602, 612 may be disposed within container 50 with second expandable body 612 positioned beneath first expandable body 602. Although a pair of expandable bodies (e.g. balloons) are shown and described herein, it should be appreciated that valve assembly 600 may include additional expandable bodies without departing from a scope of this disclosure. By way of example, first expandable body 602 may include a pair of first balloons, and second expandable body 612 may include a pair of second balloons.

Each of expandable bodies 602, 612 may include a central opening disposed through a longitudinal axis of each expandable body 602, 612. For example, first expandable body 602 may include a first central opening 608 defined by opposing interior surfaces 604, 606 of first expandable body 602 (see FIG. 9A), and second expandable body 612 may include a second central opening 618 defined by opposing interior surfaces 614, 616 of second expandable body 612 (see FIG. 9B). The longitudinal axis of each expandable body 602, 612 may be aligned with one another such that the respective central openings 608, 618 of expandable bodies 602, 612 may be in fluid communication with one another.

When valve assembly 600 is in a first position, for example as shown in FIG. 9A, second expandable body 612 may be in an inflated and/or expanded state. In this instance, the opposing interior surfaces 614, 616 of second expandable body 612 are in contact with one another to form an interface configured to close second central opening 618, thereby fluidly decoupling first chamber 52 from funnel 54 and/or second chamber 58. In the first position of valve assembly 600, first expandable body 602 may be in a (at least partially) deflated and/or unexpanded state such that the opposing interior surfaces 604, 606 of first expandable body 602 are separated from one another, thereby opening first central opening 608. In some embodiments, a volume of agent 80 within first chamber 52 may fall through (e.g. via gravity) and be received within first central opening 608 between the opposing interior surfaces 604, 606 when first expandable body 602 is in the deflated state. Agent 80 may accumulate on top of a portion of second expandable body 612, such as along the interface between the opposing interior surfaces 614, 616 of second expandable body 612 and within first central opening 608.

Referring to FIG. 9B, when actuation mechanism 30 is actuated, valve assembly 600 may be transitioned to a second position in which first expandable body 602 may be moved to an inflated and/or expand state, while second expandable body 612 may be moved to a (at least partially) deflated and/or unexpanded state. Accordingly, the volume of agent 80 received within first central opening 608 and accumulated on top of the interface between the opposing interior surfaces 614, 616 of second expandable body 612 may fall through (e.g. via gravity) and/or be received within second central opening 618. Second central opening 618 may be formed between the opposing interior surfaces 614, 616, such that agent 80 may be released into funnel 54 via second expandable body 612. Simultaneously, when valve assembly 600 is moved to the second position, an interface between the opposing interior surfaces 604, 606 of first expandable body 602 may be formed, thereby closing first central opening 608 and fluidly decoupling first chamber 52 from funnel 54 (and/or second chamber 58), such that additional agent 80 from first chamber 52 is inhibited from being released into funnel 54 by first expandable body 602.

As only a certain volume of agent 80 may be physically received within first central opening 608 of first expandable body 602 when valve assembly 600 is in the first position, valve assembly 600 may be configured to allow for a specified dose of agent 80 to be delivered to a treatment site. Accordingly, a size of first central opening 608 may be selectively adjusted (by an extent that first expandable body 602 is deflated) to accommodate for a desired dosage of agent 80, such as depending on the intended procedure. Agent 80 may be received in funnel 54 and agitated with the pressurized fluid received within second chamber 58 (and particularly funnel 54) via fluid inlet 66 as described in detail above.

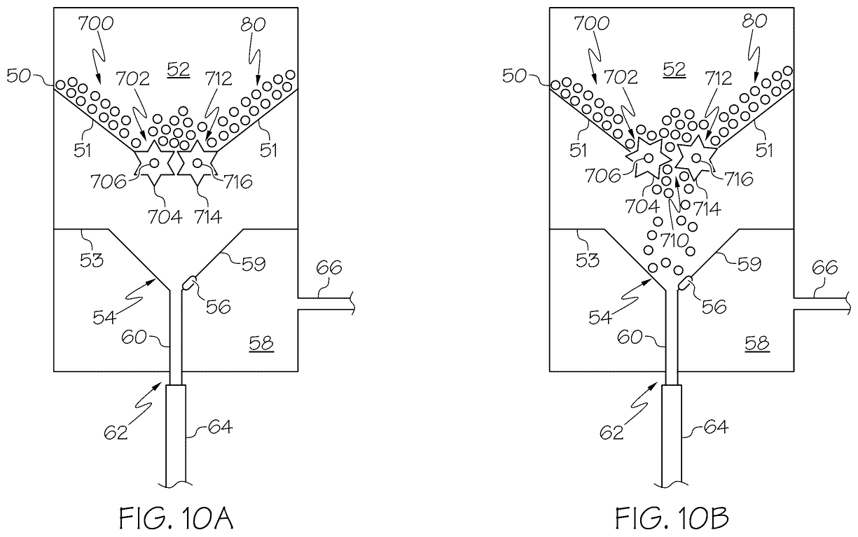

FIGS. 10A and 10B show another exemplary valve assembly 700 that may be substantially similar to valve assembly 100 except for the differences explicitly noted herein, such that like reference numerals are used to identify like components. For example, valve assembly 700 may include a first (rotatable) body 702 including a plurality of first spokes 704 extending radially outward from first body 702, and a second (rotatable) body 712 including a plurality of second spokes 714 extending radially outward from second body 712. The plurality of first spokes 704 may extend along an annular array about a perimeter of first body 702, with each first spoke 704 spaced at regular intervals about first body 702.

The plurality of second spokes 714 may extend along an annular array about a perimeter of second body 712, with each second spoke 714 spaced at regular intervals about second body 712. The plurality of first spokes 704 and the plurality of second spokes 714 may include protrusions, teeth, edges, dividers, sharpened tips, rounded tabs, and/or various other suitable mechanisms for controlling a size of a dose of agent 80 for delivery from first chamber 52 to second chamber 58. As described herein, first spokes 704 and second spokes 714 may be configured to move agent 80 from first chamber 52 to second chamber 58 and/or funnel 54. In some embodiments, spokes 704, 714 may be further configured to separate agent 80 (e.g. break agent 80 apart into smaller particles) and/or inhibit agent 80 from packing within and/or clogging first chamber 52.

Still referring to FIGS. 10A-10B, first body 702 may be configured to rotate relative to second body 712 about a pin 706, and second body 712 may be configured to rotate relative to first body 702 about a pin 716. In some embodiments, each body 702, 712 may be configured to rotate independent of one another. In other embodiments, bodies 702, 712 may be configured such that rotation of at least one of first body 702 and second body 712 may cause a corresponding rotation of the other, such as by the spokes of one body contacting and/or engaging the spokes of the other body. Bodies 702, 712 may be configured to rotate upon actuation of an actuator, such as, for example, actuation mechanism 30. In some embodiments, bodies 702, 712 may be configured to rotate in response to the pressurized fluid (released into container 50 via fluid inlet 66) applying a force thereto, such as in response to an actuation of actuation mechanism 30.

First body 702 and second body 712 may be disposed within first chamber 52 and positioned adjacent to one another such that one or more of the plurality of first spokes 704 may contact and/or abut one or more of the plurality of second spokes 714. In other embodiments, first spokes 704 may not contact second spokes 714. In the example, first body 702 may be positioned adjacent to at least one inner sidewall 51 within first chamber 52, and second body 712 may be positioned adjacent to at least one opposing inner sidewall 51 within first chamber 52. The pair of inner sidewalls 51 may be angled towards bodies 702, 712 to guide agent 80 towards each of first body 702 and second body 712.

Referring specifically to FIG. 10A, when valve assembly 700 is in a first position, one or more of the plurality of first spokes 704 of first body 702 and one or more of the plurality of second spokes 714 of second body 712 may be engaged with one another such that an interface is formed within first chamber 52 between the pair of inner sidewalls 51. In other words, at least one of the plurality of first spokes 704 and at least one of the plurality of second spokes 714 may contact one another, thereby fluidly decoupling first chamber 52 from funnel 54 and/or second chamber 58. In some embodiments, a volume of agent 80 within first chamber 52 may be received on and/or accumulate on top of the interface between the plurality of first spokes 704 and the plurality of second spokes 714. In other embodiments, first spokes 704 may be positioned adjacent to, but not in contact with, second spokes 714 such that the interface formed between first body 702 and second body 712 allows agent 80 to be collected thereon without allowing agent 80 to pass therebetween. Valve assembly 600 may be configured to inhibit movement of agent 80 from first chamber 52 to funnel 54 (and second chamber 58) when first body 702 and/or second body 712 are stationary in a fixed position.

Referring now to FIG. 10B, upon actuation of actuation mechanism 30, valve assembly 700 may be transitioned to a second position. In the second position, one or more of first body 702 and second body 712 may be configured to rotate about pins 706, 716, respectively. In some embodiments, first body 702 and second body 712 may be configured to rotate in the same direction relative to one another; while in other embodiments first body 702 and second body 712 may be configured to rotate in opposite directions relative to one another. Each of first body 702 and second body 712 may be configured to rotate in either a clockwise or a counterclockwise direction.

When valve assembly 700 is moved to the second position, one or more of first spokes 704 of first body 702 and one or more of second spokes 714 of second body 712 may become disengaged from one another, such that an opening 710 is formed between first body 702 and second body 712. Accordingly, in the second position, first chamber 52 may be fluidly coupled to funnel 54 and/or second chamber 58 via opening 710 such that agent 80 may be released into funnel 54. In some embodiments, first body 702 and second body 712 may be configured to rotate a predetermined distance and/or intervals (e.g. at least one) relative to the first position upon each actuation of actuation mechanism 30. However, first body 702 and second body 712 may alternatively, or additionally, be configured to continuously rotate so long as actuation mechanism 30 is actuated. Accordingly, in some embodiments, an initial volume of agent 80 accumulated on top of the interface between first spokes 704 and second spokes 714 may be released into funnel 54, or, alternatively, agent 80 may be continuously released into funnel 54 until actuation mechanism 30 is no longer actuated. In some embodiments, the plurality of spokes 704, 714 may be configured to divide and/or separate agent 80 into a plurality of doses as bodies 702, 712 rotate relative to one another. Agent 80 received in funnel 54 may be agitated with the pressurized fluid received within second chamber 58 (and particularly funnel 54) via fluid inlet 66 as described in detail above.

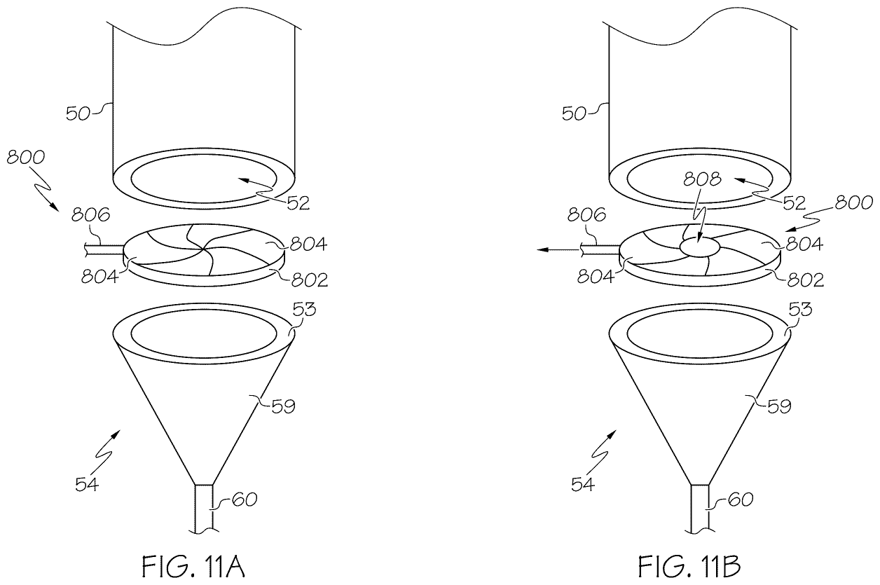

FIGS. 11A and 11B show aspects of another exemplary valve assembly 800, according to some embodiments. Valve assembly 800 may be substantially similar to valve assembly 100 except for the differences explicitly noted herein, such that like reference numerals are used to identify like components. Valve assembly 800 may include a plurality of leaves 804 positioned within and movably coupled to an outer ring 802. The plurality of leaves 804 may include various suitable mechanisms, such as walls, panels, doors, etc. In some embodiments the plurality of leaves 804 may be positioned in a spiral configuration relative to outer ring 802, and form an iris valve. However it should be appreciated that the plurality of leaves 804 may be positioned and/or arranged in various other suitable configurations than that shown and described herein without departing from a scope of this disclosure.

The plurality of leaves 804 may be configured to move relative to outer ring 802 and/or one another, such as between an extended state (FIG. 11A) and a retracted state (FIG. 11B). As described herein, the plurality of leaves 804 may be configured to engage and/or contact one another when in the extended state, and disengage one another when in the retracted state, thereby forming an opening 808 within outer ring 802. Valve assembly 800 may include an actuator 806 coupled to the plurality of leaves 804. Actuator 806 may be configured to selectively move the plurality of leaves 804 between the extended and retracted states. In some embodiments, actuator 806 may include a pull wire, a cable, a rod, etc. Actuator 806 may be coupled to one or more mechanisms of delivery system 10, such as, for example, actuation mechanism 30. Accordingly, actuator 806 may be automatically actuated in response to an actuation of actuation mechanism 30.

Referring to FIG. 11A, when valve assembly 800 is in a first position, each of the plurality of leaves 804 may extend radially inward from outer ring 802 and into opening 808 (FIG. 11B). In this instance, each of the plurality of leaves 804 may engage one or more adjacent leaves 804 within outer ring 802, thereby closing opening 808 and fluidly decoupling first chamber 52 from funnel 54 and/or second chamber 58.

When actuation mechanism 30 is actuated, valve assembly 800 may be transitioned to a second position, for example as shown in FIG. 11B. In the second position, actuator 806 (e.g., a pull wire) may be moved (e.g., pulled relative to outer ring 802), thereby moving (e.g., retracting) the plurality of leaves 804 away from a center of outer ring 802. In this instance, the plurality of leaves 804 may disengage one or more adjacent leaves 804, such that opening 808 may be formed within outer ring 802. Accordingly, first chamber 52 and funnel 54 may be fluidly coupled to one another via opening 808 and through outer ring 802.

In some embodiments, a diameter of opening 808 may vary based on an extent of movement of the plurality of leaves 804 relative to one another and/or outer ring 802. For example, the plurality of leaves 804 may be either fully retracted or partially retracted into outer ring 802. Accordingly, when the plurality of leaves 804 are partially retracted, the diameter of opening 808 may be smaller than a corresponding diameter of opening 808 when the plurality of leaves 804 are fully retracted. The variability of the diameter of opening 808 may allow for the agent stored within first chamber 52 to be released into funnel 54 at varying flow rates. Upon forming opening 808, the agent stored in first chamber 52 may be released into funnel 54 (e.g., via gravity) and agitated with the pressurized fluid received within second chamber 58 (and particularly funnel 54) via fluid inlet 66 as described in detail above.

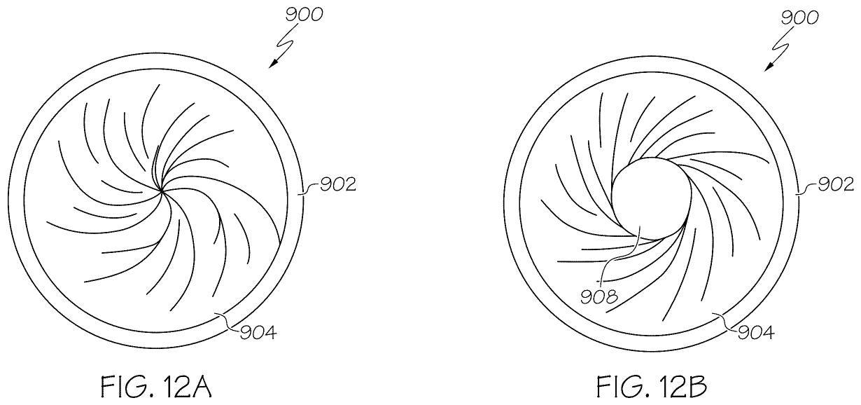

FIGS. 12A and 12B show another exemplary valve assembly 900 that may be substantially similar to valve assembly 100 except for the differences explicitly noted herein. Valve assembly 900 may include a flexible body 904 positioned within and movably coupled to an outer ring 902. In the example, flexible body 904 may form an iris valve. In some embodiments, outer ring 902 may include a pair of flanges (e.g. body halves of the outer ring) that are configured to receive flexible body 904 therebetween. The pair of flanges defining outer ring 902 may be configured to move relative to one another (e.g., away and/or towards) to transition valve assembly 900 between a first position and a second position.

Flexible body 904 may be formed of a material that is elastically deformable and/or flexible, such as a fabric, a membrane, etc. Flexible body 904 may be configured to stretch between the pair of flanges of outer ring 902, such that flexible body 904 may be configured to fold closed (FIG. 12A) and/or unfold open (FIG. 12B), depending on a separation distance between the flanges. Flexible body 904 may be configured to form an opening 908 within outer ring 902 (FIG. 12B) when moved to an unfolded state. In some embodiments, an actuator (e.g., a pull wire, a cable, a rod, etc.) may be coupled to flexible body 904 via one or more mechanical linkages for selectively expanding and/or retracting flexible body 904 relative to outer ring 902, such as in response to actuation by actuation mechanism 30. The mechanical linkages may be configured to translate an actuation of the actuator at a first degree (e.g. a first force) to a corresponding movement of flexible body 904 at a second degree (e.g. a second force) that is greater than the first degree. In this instance, a user may require less force to move flexible body 904 to the unfolded state for forming opening 908.

Referring to FIG. 12A, valve assembly 900 may be configured to move to a first position in which flexible body 904 may be in a relaxed state in which flexible body 904 extends radially inward from outer ring 902 and into opening 908. In this instance, flexible body 904 closes opening 908, thereby fluidly decoupling first chamber 52 and funnel 54. In other words, flexible body 904 is folded closed when valve assembly 900 is in the first position, such that flexible body 904 extends across an entire diameter of opening 908.

When actuation mechanism 30 is actuated, valve assembly 900 may be transitioned to a second position, for example as shown in FIG. 12B. In the second position, at least one of the flanges defining outer ring 902 may move away from the other flange of outer ring 902 to pull flexible body 904 away from the center of outer ring 902, thereby exposing opening 908 such that first chamber 52 and funnel 54 may be fluidly coupled to one another. In some embodiments, a diameter of opening 908 may be vary based on a separation distance between the pair of flanges of outer ring 902. For example, flexible body 904 may be either fully retracted or partially retracted relative to outer ring 902. Accordingly, when flexible body 904 is partially retracted, the diameter of opening 908 may be smaller than a corresponding diameter of opening 908 when flexible body 904 is fully retracted. The variability of the diameter of opening 908 may allow for the agent stored within first chamber 52 to be released into funnel 54 at varying flow rates. Upon forming opening 908, the agent stored in first chamber 52 may be released into funnel 54 (e.g., via gravity) and agitated with the pressurized fluid received within second chamber 58 (and particularly funnel 54) via fluid inlet 66 as described in detail above.

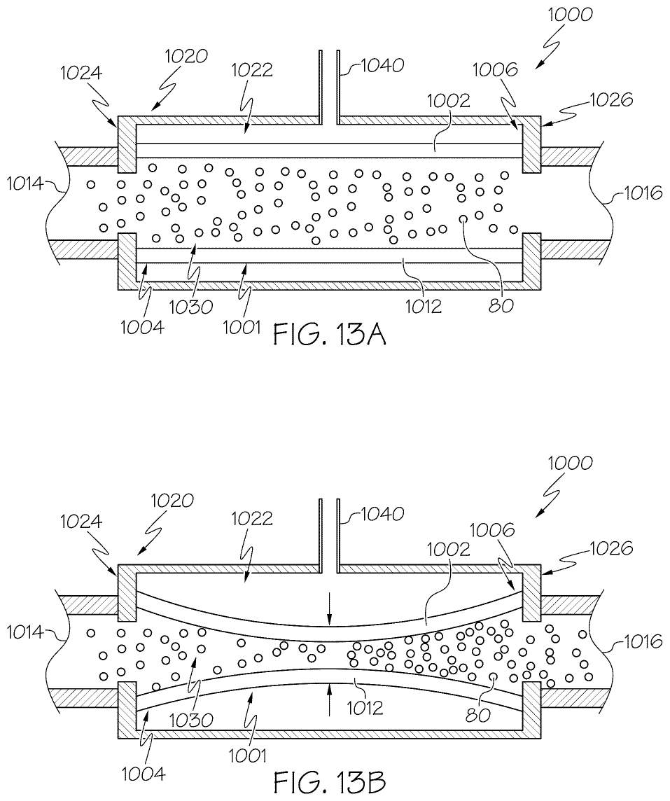

FIGS. 13A and 13B show another exemplary valve assembly 1000 that may be substantially similar to valve assembly 100 except for the differences explicitly noted herein, such that like reference numerals are used to identify like components. Valve assembly 1000 may include a conduit 1020 defining an inner cavity 1022, an inlet 1014 coupled to a first end 1024 of conduit 1020, and an outlet 1016 coupled to a second end 1026 of conduit 1020 that is opposite of first end 1024. Valve assembly 1000 may include a flexible sleeve 1001 disposed within inner cavity 1022, and flexible sleeve 1001 may include an upper portion 1002 and a lower portion 1012 positioned opposite of upper portion 1002. It should be appreciated that upper portion 1002 and lower portion 1012 may be readily interchangeable with one another without departing from a scope of this disclosure.

Flexible sleeve 1001 may have various suitable sizes and/or shapes, such as a cylindrical body defined by upper portion 1002 and lower portion 1012. Flexible sleeve 1001 may have a longitudinal length defined between a first end 1004 and a second end 1006 that is positioned opposite of first end 1004. Flexible sleeve 1001 may have a lumen 1030 defined collectively by upper portion 1002 and lower portion 1012, and lumen 1030 may extend between first end 1004 and second end 1006. First end 1004 of flexible sleeve 1001 may be fixedly coupled to first end 1024 of conduit 1020, such that first end 1004 is securely fixed within inner cavity 1022 to first end 1024. First end 1004 may be in fluid communication with inlet 1014, and inlet 1014 may be in further fluid communication with first chamber 52 (see FIG. 1).

Still referring to FIGS. 13A-13B, second end 1006 of flexible sleeve 1001 may be fixedly coupled to second end 1026 of conduit 1020, such that second end 1006 is securely fixed within inner cavity 1022 to second end 1026. Second end 1006 may be in fluid communication with outlet 1016, and outlet 1016 may be in further fluid communication with funnel 54 and/or second chamber 58 (see FIG. 1). In some embodiments, flexible sleeve 1001 may be configured to receive agent 80 from inlet 1014, such that agent 80 may enter flexible sleeve 1001 through first end 1004. As described herein, valve assembly 1000 may be configured to urge agent 80 through flexible sleeve 1001 and out of second end 1006 for delivery through outlet 1016.

Flexible sleeve 1001 may be formed of various flexible and/or deformable materials, including but not limited to, rubber. Valve assembly 1000 may include a fluid inlet 1040 that is fluidly coupled to the pressurized medium source of delivery system 10 (see FIG. 1) and conduit 1020. Accordingly, the pressurized fluid from the pressurized medium source may be delivered into inner cavity 1022 via fluid inlet 1040. It should be appreciated that fluid inlet 1040 may be fluidly coupled to conduit 1020 at various suitable locations, walls, ends, and/or surfaces without departing from a scope of this disclosure. In other embodiments, fluid inlet 1040 may be coupled upstream of conduit 1020, such as adjacent to inlet 1014.

When valve assembly 1000 is in a first position, for example as shown in FIG. 13A, actuation mechanism 30 may be unactuated such that pressurized fluid is not actively delivered into inner cavity 1022 via fluid inlet 1040. Accordingly, flexible sleeve 1001 may be maintained in an expanded and/or uncompressed state such that upper portion 1002 and lower portion 1012 are separated from one another. Agent 80 from inlet 1014 may be received through first end 1004 and stored within lumen 1030 when flexible sleeve 1001 is in the uncompressed state.

Referring now to FIG. 13B, a pressurized fluid may be delivered into conduit 1020 via fluid inlet 1040 to transition valve assembly 1000 to the second position. In this instance, the pressurized fluid may be received about an exterior of flexible sleeve 1001, and particularly about upper portion 1002 and lower portion 1012. As a result, the pressurized fluid may push upper portion 1002 and lower portion 1012 radially inwards toward one another, causing flexible sleeve 1001 to compress. In other words, upper portion 1002 and lower portion 1012 of flexible sleeve 1001 may be pinched together, thereby decreasing a diameter of lumen 1030 and urging agent 80 out of lumen 1030 via second end 1006 and through outlet 1016. Agent 80 received in funnel 54 from outlet 1016 may be agitated with the pressurized fluid received within second chamber 58 (and particularly funnel 54) via fluid inlet 66 as described in detail above. It should be appreciated that the pressurized medium source of delivery system 10 may simultaneously deliver pressurized fluid to conduit 1020 (via fluid inlet 1040), for delivering agent 80 towards funnel 54, and to funnel 54 for agitating agent 80 received therein from flexible sleeve 1001.

Alternatively, in some embodiments, valve assembly 1000 may include an actuator (e.g., a cord, a wire, a rod, etc.) coupled to flexible sleeve 1001 for moving valve assembly 1000 from the first position to the second position. For example, the actuator may extend into conduit 1020 via fluid inlet 1040 and be wrapped around an exterior of flexible sleeve 1001. In this instance, upon actuation of the actuator (e.g. pulling the actuator), the actuator (e.g., a cord, a wire, a rod, etc.) may mechanically compress flexible sleeve 1001. In some embodiments, the actuator (e.g., a cord or a wire) may be encased in flexible sleeve 1001, and may include an end extending out of one or more of first end 1004 and/or second end 1006 of flexible sleeve 1001. Upon actuation of actuation mechanism 30, the actuator may be pulled to cause flexible sleeve 1001 to compress. In this instance, the actuator may function as a drawstring to cinch flexible sleeve 1001 closed.

In other embodiments, the actuator (e.g., a wire, a cord, etc.) may be formed of a shape-memory material (e.g., Nitinol) that is configured to automatically return valve assembly 1000 to the first position by urging flexible sleeve 1001 to the expanded state upon the user releasing the actuator. In further embodiments, valve assembly 1000 may include a biasing mechanism (e.g., a spring) coupled to flexible sleeve 1001, the biasing mechanism being configured to bias valve assembly 1000 to the first position and/or the second position. Accordingly, the biasing mechanism may return flexible sleeve 1001 to the expanded state after being compressed by actuation of the actuator, and vice versa.

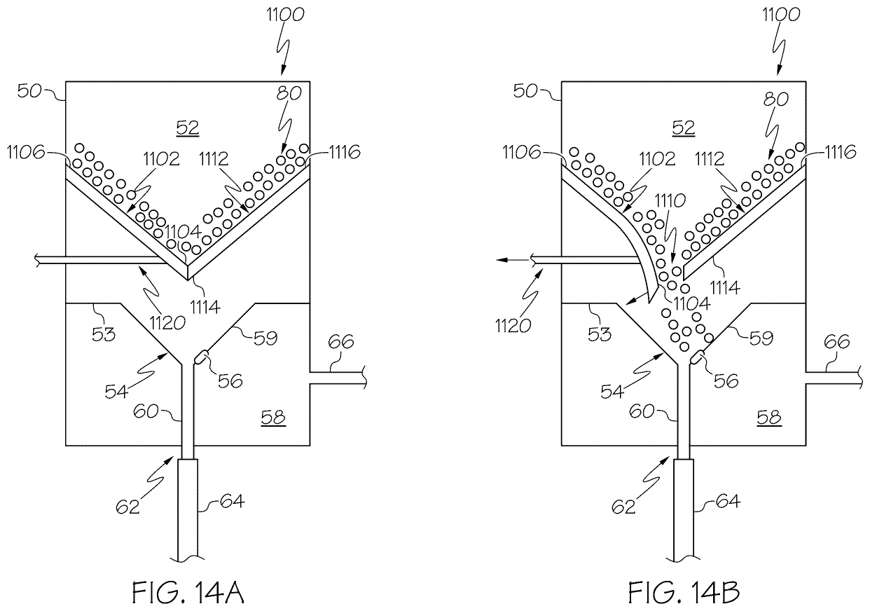

FIGS. 14A and 14B show aspects of another exemplary valve assembly 1100, according to some embodiments. Valve assembly 1100 may be substantially similar to valve assembly 100 such that like reference numerals are used to identify like components. Valve assembly 1100 may include a first body 1102 that is at least partially deformable, and a second body 1112 that is rigid relative to first body 1102. First body 1102 may include a first end 1104 and a second end 1106 that is opposite of first end 1104. Second body 1112 may include a first end 1114 and a second end 1116 that is opposite of first end 1114. Bodies 1102, 1112 may each be disposed within container 50, and coupled to an interior surface of container 50 along second ends 1106, 1116, respectively. Bodies 1102, 1112 may define internal sidewalls of first chamber 52, such that agent 80 stored in first chamber 52 may be at least partially received along an exterior surface of bodies 1102, 1112.

First body 1102 may be formed of a flexible and/or deformable material, including but not limited to, silicone. First end 1104 may be configured to move relative to second end 1106, thereby forming an opening 1110 between first body 1102 and second body 1112 when valve assembly 1100 moves to a second position (see FIG. 14B). In some embodiments, valve assembly 1100 may include an actuator, such as a piston 1120, coupled to first body 1102. Piston 1120 may be coupled to first body 1102 adjacent to first end 1104. Piston 1120 may be configured to flexibly deform first body 1102 upon actuation, thereby moving first end 1104 laterally away from first end 1114 of second body 1112. In some embodiments, first body 1102 and/or piston 1120 may be biased to a first position (FIG. 14A) with first end 1104 maintained in contact with first end 1114 of second body 1112. In other embodiments, valve assembly 1100 may include a biasing mechanism (e.g., a spring) coupled to first body 1102 and/or piston 1120 to bias first end 1104 towards the first position.

Referring specifically to FIG. 14A, when valve assembly 1100 is in a first position, first end 1104 of first body 1102 may be configured to contact first end 1114 of second body 1112, thereby forming an interface between first end 1104 of first body 1102 and first end 1114 of second body 1112. In other words, first body 1102 may be configured to form a fluid tight seal with second body 1112 in response to first end 1104 abutting against first end 1114. In this instance, bodies 1102, 1112 may be configured to fluidly decouple first chamber 52 from second chamber 58 and/or funnel 54. Accordingly, agent 80 stored within first chamber 52 may be inhibited from being released into funnel 54 when valve assembly 1100 is in the first position.

When actuation member 30 is actuated, valve assembly 1100 may be moved to a second position, for example as shown in FIG. 14B. Piston 1200 may move relative to container 50, thereby pulling first body 1102 away from second body 1112, such as towards an inner surface of container 50 that is positioned opposite of second body 1112. In response, first body 1102 may flex radially-outward away from second body 1112, thereby creating opening 1110 between first end 1104 of first body 1102 and first end 1114 of second body 1112. It should be appreciated that first body 1102 may be deformed to various shapes and/or configurations as valve assembly 1100 moves to the second position, such as, for example, a convex or concave configuration.

In this instance, with first end 1104 disengaged from first end 1114, first body 1102 may be configured to fluidly couple first chamber 52 with funnel 54 such that agent 80 stored within first chamber 52 may be released into funnel 54. Once actuation mechanism 30 is disengaged, piston 1120 may be configured to release and/or push first body 1102 back to the first position in which first end 1104 contacts first end 1114, thereby closing opening 1110 and fluidly decoupling first chamber 52 from funnel 54. As described above, valve assembly 1100 may include a biasing mechanism in some embodiments, and the biasing mechanism may be configured to bias piston 1120 to an initial position, thereby urging first body 1102 towards second body 1112 once actuation mechanism 30 is no longer actuated.

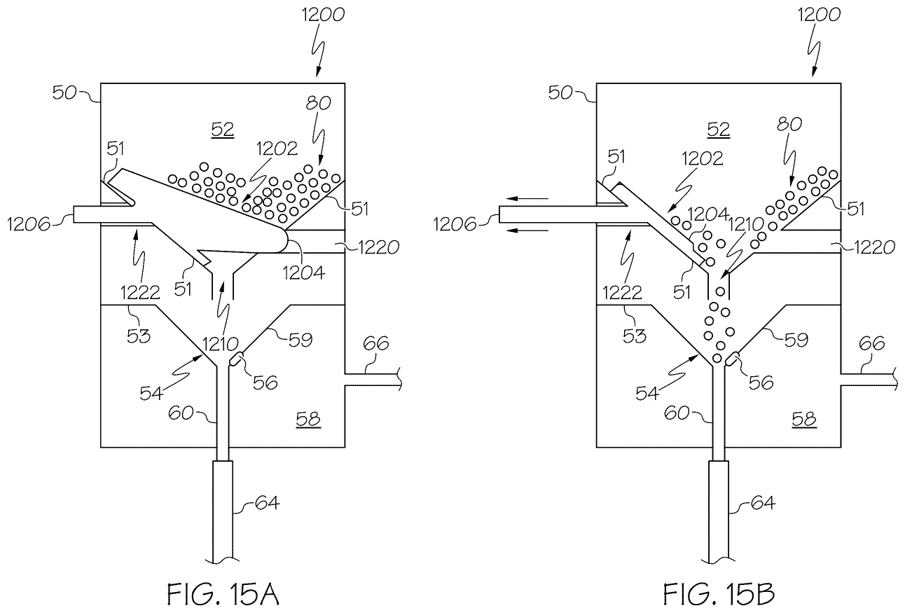

FIGS. 15A and 15B show another exemplary valve assembly 1200 that may be substantially similar to valve assembly 100 such that like reference numerals are used to identify like components. Valve assembly 1200 may include a deformable body 1202 (e.g., a flexible plug) having at least a first end 1204 and at least a second end 1206. Deformable body 1202 may be disposed within first chamber 52 with at least a portion of deformable body 1202 covering an opening 1210 defined between the pair of inner sidewalls 51 of container 50. Accordingly, deformable body 1202 may be configured to fluidly decouple first chamber 52 from funnel 54 when opening 1210 is closed by deformable body 1202. In some embodiments, at least a portion of deformable body 1202 may form a plug that seals opening 1210, such as an intermediary portion of deformable body 1202 between first end 1204 and second end 1206.

Container 50 may include a first channel or cavity 1220 along at least one of the pair of inner sidewalls 51, and a second channel or cavity 1222 along at least another one of the pair of inner sidewalls 51. First end 1204 may be configured to extend at least partially into first cavity 1220, and second end 1206 may be configured to extend at least partially into second cavity 1222. In one example, first cavity 1220 may be fluidly coupled to the pressurized medium source of delivery system 10 (see FIG. 1). In this instance, container 50 may be configured to receive a pressurized medium within first chamber 52 via first cavity 1220. The pressurized fluid received therein may be configured to urge first end 1204 out of first cavity 1220, and further urge second end 1206 further into second cavity 1222. Stated differently, first end 1204 may be moved out of first cavity 1220 in response to the pressurized medium encountering first end 1204 within first cavity 1220. In response to first end 1204 moving relative to first chamber 52, deformable body 1202 may be deformed and/or moved in a direction away from first cavity 1220 and towards second cavity 1222. In this instance, second end 1206 may be urged further into second cavity 1222 in response to the deformation and/or movement of deformable body 1202 by the pressurized medium, thereby uncovering opening 1210.

In another example, valve assembly 1200 may include an actuator (not shown) coupled to second end 1206 within second cavity 1222. The actuator, which may be coupled to actuation mechanism 30, may be configured to at least partially deform deformable body 1202 in response to moving (e.g., pulling) second end 1206 relative to second cavity 1222. Stated differently, second end 1206 may define a stem of deformable body 1202 that may be coupled to the actuator (e.g. a pull wire, a cable, a rod, etc.). Second end 1206 may be pulled and/or stretched in response to an actuation of the actuator (not shown), thereby deforming deformable body 1202 by extending second end 1206 and/or an intermediary portion of deformable body 1202 further into second cavity 1222. In this instance, first end 1204 may be pulled out of first cavity 1220, thereby uncovering opening 1210. Deformable body 1202 may be made from various suitable materials, including a shape memory material, such as, for example, a liquid silicone rubber. Accordingly, deformable body 1202 may be configured to return to a first shape and/or configuration (see FIG. 15A) after being deformed to a second shape and/or configuration (see FIG. 15B), as described in further detail herein.

Referring specifically to FIG. 15A, when valve assembly 1200 is in a first position, deformable body 1202 may extend across opening 1210, thereby fluidly decoupling first chamber 52 from funnel 54 and/or second chamber 58. First end 1204 may be configured to seal first cavity 1220 when valve assembly 1200 is in the first position, such that first chamber 52 may be fluidly decoupled from the pressurized medium source that is in fluid communication with first cavity 1220.

Referring now to FIG. 15B, upon actuation of actuation mechanism 30, valve assembly 1200 may be configured to move to the second position. Second end 1206 of deformable body 1202 may be moved through second cavity 1222 and first end 1204 may be moved out of first cavity 1220, which may cause deformable body 1202 to collapse and/or change shape from a first (original) shape (FIG. 15A) to a second (collapsed) shape. In other words, with second end 1206 extending further into second cavity 1222, first end 1204 may be at least partially retracted from first cavity 1220, thereby uncovering opening 1210 at the bottom of first chamber 52. As detailed above, deformable body 1202 may be configured to move to the second position upon the pressurized medium urging (pushing) first end 1204 and second 1206 to move relative to first cavity 1220 and second cavity 1222, respectively. Alternatively, deformable body 1202 may be configured to move to the second position upon the actuator urging (pulling) second end 1206 relative to second cavity 1222, thereby pulling first end 1204 relative to first cavity 1220.

Accordingly, in the second position of valve assembly 1200, first chamber 52 may be fluidly coupled to funnel 54 such that agent 80 stored within first chamber 52 may be released into funnel 54 via opening 1210. Once actuation mechanism 30 is disengaged, first end 1204 may no longer be urged by the pressurized medium delivered through first cavity 1220, thereby returning deformable body 1202 to the first (original) shape. Alternatively, once actuation mechanism 30 is disengaged, second end 1206 of deformable body 1202 may be released by the actuator coupled thereto within second cavity 1222, thereby returning deformable body 1202 to the first (original) shape. In either instance, deformable body 1202 may return close opening 1210 when valve assembly 1200 returns to the first position.

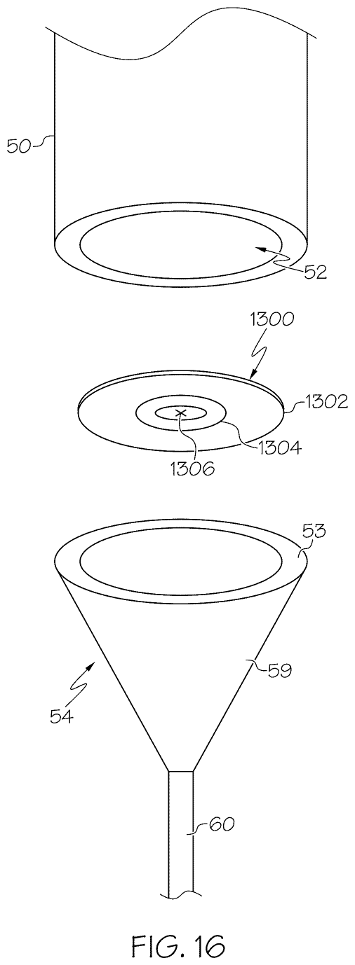

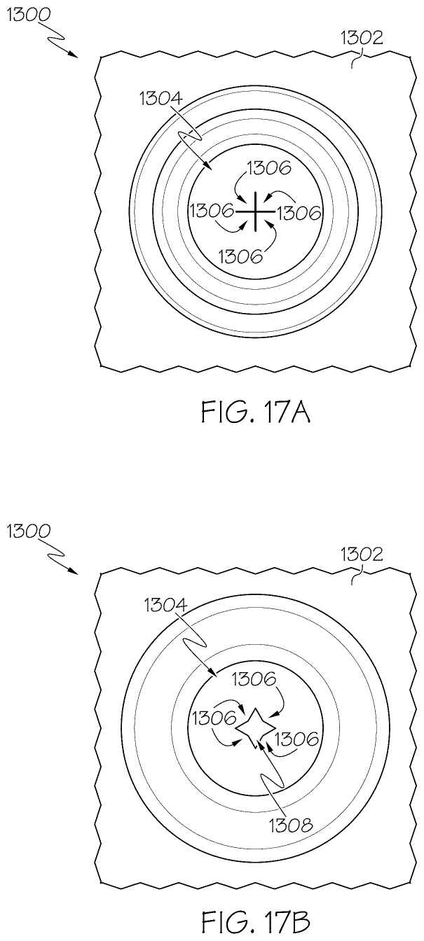

FIGS. 16-17B show another exemplary valve assembly 1300 that may be substantially similar to valve assembly 100 except for the differences explicitly noted herein. Accordingly, like reference numerals are used to identify like components. Valve assembly 1300 may include a body 1302 including a seal 1304 formed of a flexible and/or elastomeric material that is at least partially deformable. Valve assembly 1300 may be positioned between first chamber 52 and funnel 54. In some embodiments, an upper surface and/or interior cavity of body 1302 may be configured to receive a volume of the agent stored within first chamber 52 (e.g., via gravity) thereon.

Seal 1304 may include one or more (e.g., a plurality) of slits 1306 (e.g., panels, walls, flaps, surfaces, etc.) that are configured to abut and/or contact one another when valve assembly 1300 is in a first position (FIG. 17A). Further, the plurality of slits 1306 may be configured to move relatively away from one another when valve assembly 1300 is in a second position (FIG. 17B). The plurality of slits 1306 may be configured to form a fluid tight seal between first chamber 52 and second chamber 58 when valve assembly 1300 is in the first position (see FIG. 17B), and form an opening 1308 through seal 1304 when valve assembly 1300 is in the second position (see FIG. 17B).

As described herein, valve assembly 1300 may be configured to transition from the first position to the second position in response to container 50 receiving a pressurized medium within first chamber 52 and/or second chamber 58, and the pressurized medium applying a force against seal 1304 causing the plurality of slits 1306 to move relative to one another. In the example, four slits 1306 are formed within seal 1304, however, it should be appreciated that additional and/or fewer slits 1306 may be included along seal 1304 without departing from a scope of this disclosure.

Referring now to FIG. 17A, when valve assembly 1300 is in a first position, the plurality of slits 1306 may abut one another such that seal 1304 forms a fluid tight seal between first chamber 52 and second chamber 58. In other words, first chamber 52 may be fluidly decoupled from funnel 54 when the plurality of slits 1306 are in contact with one another. The plurality of slits 1306 may be configured to close opening 1308 at seal 1304 absent the application of a force and/or a pressure onto body 1302.

Referring to FIG. 17B, when actuation mechanism 30 is actuated, valve assembly 1300 may be transitioned to a second position in response to a pressurized medium being delivered into container 50, such as in first chamber 52 and/or second chamber 58. In this instance, an increased pressure may be applied to an upper surface of body 1302 (adjacent to first chamber 52) by the pressurized medium received within container 50, thereby causing each of the plurality of slits 1306 to extend outwardly away from one another to form opening 1308 along seal 1304. Agent 80 stored within first chamber 52 may be released into funnel 54 via opening 1308 while the pressurized medium is continuously delivered into first chamber 52, exerting a continuous force and/or pressure against seal 1304.

Upon ceasing actuation of actuation mechanism 30, the plurality of slits 1306 may move back into contact with one another as the force and/or pressure applied to seal 1304 by the pressurized medium is removed. In this instance, the plurality of slits 1306 are configured to return to an original position, thereby returning valve assembly 1300 to the first position (FIG. 17A). In this instance, body 1302 may be configured to close opening 1308, thereby fluidly decoupling first chamber 52 from funnel 54.

While principles of this disclosure are described herein with reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

We claim:

1. A valve assembly for a medical device, comprising:
an enclosure configured to store an agent;
a funnel coupled to the enclosure, and configured to receive the agent via an opening of the enclosure; and
a valve fluidly coupled to the enclosure and the funnel, wherein the valve is at least partially disposed between the enclosure and the funnel, and at least a portion of the agent is received on the valve;
wherein the valve is configured to move from a first position to a second position relative to the enclosure and the funnel to selectively release the agent from the enclosure into the funnel;
wherein, in the first position, the valve is configured to close the opening and inhibit the agent from exiting the enclosure and entering the funnel, and in the second position, the valve is configured to open the opening and release the agent from the enclosure for delivery into the funnel.

2. The valve assembly of claim 1, wherein the valve includes a body movably disposed within the opening;
wherein, in the first position, at least a portion of the body is configured to extend into the opening, thereby fluidly decoupling the enclosure from the funnel, and in the second position, at least the portion of the body is configured to retract out of the opening, thereby fluidly coupling the enclosure with the funnel.

3. The valve assembly of claim 2, wherein the valve includes a hinge coupled to the body and a protrusion extending outwardly from the body;
wherein the body is configured to pivot about the hinge when moving from the first position to the second position, and the protrusion is configured to extend into the enclosure when the body is in the first position and into the funnel when the body is in the second position.

4. The valve assembly of claim 1, wherein the valve includes a first body having one or more first ends and a second body having one or more second ends, the second body is positioned opposite of the first body within the enclosure;
wherein, in the first position, the first body is configured to engage the second body by interlocking the one or more first ends with the one or more second ends, and in the second position, the first body is configured to disengage the second body by separating the one or more first ends from the one or more second ends.

5. The valve assembly of claim 1, wherein the valve defines at least one wall of a plurality of walls of the enclosure, such that the at least one wall is configured to move relative to the remaining walls of the plurality of walls from the first position to the second position to form the opening.

6. The valve assembly of claim 1, further including a channel positioned between the enclosure and the funnel, wherein the valve includes a rod that is movable relative to the channel;
wherein, in the first position, the rod is configured to extend outwardly from the channel and into the opening of the enclosure, and in the second position, the rod is configured to retract into the channel and out of the opening.

7. The valve assembly of claim 1, wherein the valve includes a first expandable body positioned between the enclosure and the funnel, and a second expandable body positioned between the first expandable door and the funnel, wherein each of the first expandable body and the second expandable body include a center opening;
wherein, in the first position, the first expandable body is at least partially unexpanded such that the center opening of the first expandable body is open to receive a first portion of the agent from the enclosure, and the second expandable body is expanded such that the center opening of the second expandable body is closed to inhibit the first portion of the agent received through the first expandable body from entering the funnel.

8. The valve assembly of claim 1, wherein:
the valve includes a first rotatable body and a second rotatable body, each of the first rotatable body and the second rotatable body includes a plurality of projections;
in the first position, a first projection of the first rotatable body and a first projection of the second rotatable body are configured to engage one another and form an interface for collecting a portion of the agent from the enclosure; and
in the second position, the first projection of the first rotatable body and the first projection of second rotatable body are configured to disengage one another to open the interface between the first projection of the first rotatable body and the first projection of the second rotatable body for releasing the portion of the agent into the funnel.

9. The valve assembly of claim 1, wherein:
the valve includes a ring defining an opening and a plurality of leaves movably disposed within the opening;
in the first position, each of the plurality of leaves is extended radially inward from the ring and into the opening, such that each of the plurality of leaves is configured to engage one another and close the opening; and
in the second position, each of the plurality of leaves is configured to retract radially outward from the opening, such that each of the plurality of leaves is configured to disengage one another and open the opening.

10. The valve assembly of claim 1, wherein:
the valve includes a first flange, a second flange, and a flexible fabric coupled between the first flange and the second flange, the first flange and the second flange collectively define a center opening and the flexible fabric is disposed within the center opening;
in the first position, at least one of the first flange and the second flange is configured to move towards the other to transition the flexible fabric to a first configuration in which the flexible fabric closes the center opening; and
in the second position, at least one of the first flange and the second flange is configured to move away from the other to transition the flexible fabric to a second configuration in which the flexible fabric opens the center opening.

11. The valve assembly of claim 1, wherein the valve includes a conduit defining a channel and a flexible sleeve disposed within the channel, the conduit is configured to receive at least a portion of the agent from the enclosure;
wherein, in the first position, the flexible sleeve is configured to move radially outward relative to the channel to a first configuration in which the flexible sleeve opens the conduit for receiving the agent from the enclosure; and
wherein, in the second position, the flexible sleeve is configured to move radially inward relative to the channel to a second configuration in which the flexible sleeve closes the conduit and urges the agent out of the channel and towards the funnel.

12. The valve assembly of claim 11, wherein the valve includes a wire assembly coupled to the flexible sleeve, the wire assembly is configured to move the flexible sleeve relative to the conduit between the first configuration and the second configuration.

13. The valve assembly of claim 1, wherein the valve includes a deformable body configured to extend into the opening of the enclosure;
wherein, in the first position, the deformable body is configured to maintain a first shape that closes the opening of the enclosure, and in the second position, the deformable body is configured to transition to a second shape that opens the opening of the enclosure.

14. The valve assembly of claim 1, wherein the valve includes a deformable body coupled to the opening of the enclosure;
wherein, in the first position, the deformable body is configured to cover the opening of the enclosure, and in the second position, the deformable body is configured to flex radially outward away from the opening of the enclosure, thereby allowing the agent to exit the enclosure and enter the funnel.

15. The valve assembly of claim 1, wherein the valve includes a seal including a plurality of slits;
wherein, in the first position, each of the plurality of slits is configured to abut one another, thereby closing the opening of the enclosure, and in the second position, each of the plurality of slits is configured to extend outwardly away from one another such that the opening is formed through the seal for receiving the agent.

16. A valve assembly for a medical device, comprising:
an enclosure defining a first chamber configured to store an agent and a second chamber configured to receive a pressurized fluid; and
a valve disposed within the enclosure and positioned between the first chamber and the second chamber, wherein the valve is configured to fluidly couple the first chamber to the second chamber via an opening formed by the valve;
wherein the valve is configured to move relative to the enclosure to a first position to close the opening between the first chamber and the second chamber, thereby inhibiting the agent from exiting the first chamber and entering the second chamber such that the pressurized fluid is not in fluid communication with the agent; and
wherein the valve is configured to move relative to the enclosure to a second position to open the opening between the first chamber and the second chamber, thereby allowing the agent to exit the first chamber and enter the second chamber such that the pressurized fluid is in fluid communication with the agent.

17. The valve assembly of claim 16, wherein the valve includes a first expandable body positioned between the first chamber and the second chamber, and a second expandable body positioned between the first expandable body and the second chamber, the opening is at least partially defined by each of the first expandable body and the second expandable body;
wherein, in the first position, the first expandable body is at least partially compressed and the second expandable body is expanded, such that a first portion of the agent from the first chamber is received through the first expandable body and not through the second expandable body; and
wherein, in the second position, the first expandable body is expanded and the second expandable body is at least partially compressed, such that the first portion of the agent received through the first expandable body is received through the second expandable door and released into the second chamber via the opening.

18. The valve assembly of claim 17, wherein, in the first position, the second expandable body is configured to inhibit the first portion of the agent received through the first expandable body from entering the second chamber via the opening; and
wherein, in the second position, the first expandable body is configured to inhibit a second portion of the agent from the first chamber from entering through the second expandable body via the opening.

19. The valve assembly of claim 16, wherein the valve includes an iris valve formed of a flexible fabric that is configured to move laterally relative to the enclosure to form the opening between the first chamber and the second chamber.

20. A method for delivering an agent from a medical device, the medical device including an enclosure having a first chamber storing the agent and a second chamber that is in fluid communication with a pressurized fluid;
the method comprising:
actuating the medical device to deliver the pressurized fluid to the second chamber of the enclosure;
moving a valve of the medical device from a first position to a second position in response to the pressurized fluid being delivered to the second chamber, wherein, in the first position, the valve is configured to seal the first chamber from the second chamber such that the agent is inhibited from exiting the first chamber, and in the second position, the valve is configured to unseal the first chamber from the second chamber such that the agent is permitted to exit the first chamber and enter the second chamber;
agitating the agent with the pressurized fluid within the second chamber, thereby forming a mixture of the agent and the pressurized fluid; and
guiding the mixture of the agent and the pressurized fluid through an outlet of the medical device.
